SEQUENCE LISTING (24 sequences)

SEQ ID NO: 1 - RNA, 23 nt, synthetic construct
  misc_feature 1..23: note = Synthetic: miR-17
  source 1..23: mol_type = other RNA
caaagtgcttacagtgcaggtag

SEQ ID NO: 2 - RNA, 23 nt, synthetic construct
  misc_feature 1..23: note = Synthetic: miR-20a
  source 1..23: mol_type = other RNA
taaagtgcttatagtgcaggtag

SEQ ID NO: 3 - RNA, 23 nt, synthetic construct
  misc_feature 1..23: note = Synthetic: miR-20b
  source 1..23: mol_type = other RNA
caaagtgctcatagtgcaggtag

SEQ ID NO: 4 - RNA, 23 nt, synthetic construct
  misc_feature 1..23: note = Synthetic: miR-93
  source 1..23: mol_type = other RNA
caaagtgctgttcgtgcaggtag

SEQ ID NO: 5 - RNA, 23 nt, synthetic construct
  misc_feature 1..23: note = Synthetic: miR-106a
  source 1..23: mol_type = other RNA
aaaagtgcttacagtgcaggtag

SEQ ID NO: 6 - RNA, 21 nt, synthetic construct
  misc_feature 1..21: note = Synthetic: miR-106b
  source 1..21: mol_type = other RNA
taaagtgctgacagtgcagat

SEQ ID NO: 7 - RNA, 11 nt, synthetic construct
  misc_feature 1..11: note = Synthetic: RG-NG-1004
  modified_base 1..11: mod_base = OTHER; note = S-cEt nucleosides
  source 1..11: mol_type = other RNA
taagcactttg

SEQ ID NO: 8 - RNA, 11 nt, synthetic construct
  misc_feature 1..11: note = Synthetic: RG-NG-1005
  modified_base 1..2: mod_base = OTHER; note = S-cEt nucleosides
  modified_base 3: mod_base = OTHER; note = 2'-O-methyl nucleoside
  modified_base 5..7: mod_base = OTHER; note = S-cEt nucleosides
  modified_base 8..9: mod_base = OTHER; note = 2'-O-methyl nucleosides
  modified_base 10..11: mod_base = OTHER; note = S-cEt nucleosides
  source 1..11: mol_type = other RNA
taagcactttg

SEQ ID NO: 9 - RNA, 15 nt, synthetic construct
  modified_base 3: mod_base = OTHER; note = thymine
  modified_base 5: mod_base = OTHER; note = thymine
  misc_feature 1..15: note = Synthetic: RG-NG-1018
  modified_base 1..3: mod_base = OTHER; note = 2'-O-methoxyethyl
  modified_base 4: mod_base = OTHER; note = S-cEt nucleoside
  modified_base 5: mod_base = OTHER; note = 2'-O-methoxyethyl
  modified_base 7..8: mod_base = OTHER; note = S-cEt nucleoside
  modified_base 9: mod_base = OTHER; note = 2'-O-methyl nucleoside
  modified_base 10..12: mod_base = OTHER; note = 2'-fluoro nucleoside
  modified_base 13: mod_base = OTHER; note = 2'-O-methyl nucleoside
  modified_base 14..15: mod_base = OTHER; note = S-cEt nucleoside
  source 1..15: mol_type = other RNA
actgtaagcactttc

SEQ ID NO: 10 - RNA, 15 nt, synthetic construct
  modified_base 3: mod_base = OTHER; note = thymine
  misc_feature 1..15: note = Synthetic: RG-NG-1019
  modified_base 1..2: mod_base = OTHER; note = S-cEt nucleosides
  modified_base 4..5: mod_base = OTHER; note = S-cEt nucleosides
  modified_base 7..8: mod_base = OTHER; note = S-cEt nucleosides
  modified_base 9: mod_base = OTHER; note = 2'-O-methyl nucleoside
  modified_base 10..12: mod_base = OTHER; note = 2'-fluoro nucleosides
  modified_base 13: mod_base = OTHER; note = 2'-O-methyl nucleosides
  modified_base 14..15: mod_base = OTHER; note = S-cEt nucleosides
  source 1..15: mol_type = other RNA
actgtaagcactttc

SEQ ID NO: 11 - RNA, 13 nt, synthetic construct
  modified_base 1: mod_base = OTHER; note = thymine
  modified_base 3: mod_base = OTHER; note = thymine
  misc_feature 1..13: note = Synthetic: RG-NG-1020
  modified_base 1: mod_base = OTHER; note = 2'-O-methoxyethyl
  modified_base 2: mod_base = OTHER; note = S-cEt nucleoside
  modified_base 3: mod_base = OTHER; note = 2'-O-methoxyethyl
  modified_base 5..6: mod_base = OTHER; note = S-cEt nucleoside
  modified_base 7: mod_base = OTHER; note = 2'-O-methyl nucleoside
  modified_base 8..10: mod_base = OTHER; note = 2'-fluoro nucleosides
  modified_base 11: mod_base = OTHER; note = 2'-O-methyl nucleoside
  modified_base 12..13: mod_base = OTHER; note = S-cEt nucleoside
  source 1..13: mol_type = other RNA
tgtaagcactttc

SEQ ID NO: 12 - RNA, 11 nt, synthetic construct
  modified_base 1: mod_base = OTHER; note = thymine
  misc_feature 1..11: note = Synthetic: RG-NG-1021
  modified_base 1: mod_base = OTHER; note = 2'-O-methoxyethyl
  modified_base 3..4: mod_base = OTHER; note = S-cEt nucleoside
  modified_base 5: mod_base = OTHER; note = 2'-O-methyl nucleoside
  modified_base 6..8: mod_base = OTHER; note = 2'-fluoro nucleosides
  modified_base 9: mod_base = OTHER; note = 2'-O-methyl nucleoside
  modified_base 10..11: mod_base = OTHER; note = S-cEt nucleoside
  source 1..11: mol_type = other RNA
taagcactttc

SEQ ID NO: 13 - RNA, 11 nt, synthetic construct
  modified_base 1: mod_base = OTHER; note = thymine
  misc_feature 1..11: note = Synthetic: RG-NG-1022
  modified_base 1..2: mod_base = OTHER; note = 2'-O-methoxyethyl
  modified_base 3..4: mod_base = OTHER; note = S-cEt nucleoside
  modified_base 5: mod_base = OTHER; note = 2'-O-methyl nucleoside
  modified_base 6..8: mod_base = OTHER; note = 2'-fluoro nucleosides
  modified_base 9: mod_base = OTHER; note = 2'-O-methyl nucleoside
  modified_base 10..11: mod_base = OTHER; note = S-cEt nucleoside
  source 1..11: mol_type = other RNA
taagcactttc

SEQ ID NO: 14 - RNA, 10 nt, synthetic construct
  modified_base 8: mod_base = OTHER; note = thymine
  misc_feature 1..10: note = Synthetic: RG-NG-1030
  modified_base 1..2: mod_base = OTHER; note = 2'-O-methyl nucleosides
  modified_base 3..6: mod_base = OTHER; note = LNA
  modified_base 7: mod_base = OTHER; note = 2'-O-methyl nucleosides
  modified_base 8: mod_base = OTHER; note = LNA
  modified_base 10: mod_base = OTHER; note = 2'-O-methyl nucleosides
  source 1..10: mol_type = other RNA
aagcactttc

SEQ ID NO: 15 - RNA, 11 nt, synthetic construct
  misc_feature 1..11: note = Synthetic: RG-NG-1004
  modified_base 1..11: mod_base = OTHER; note = S-cEt nucleosides
  source 1..11: mol_type = other RNA
taagcactttg

SEQ ID NO: 16 - RNA, 11 nt, synthetic construct
  misc_feature 1..11: note = Synthetic: RG-NG-1005
  modified_base 1..2: mod_base = OTHER; note = S-cEt nucleosides
  modified_base 3: mod_base = OTHER; note = 2'-O-methyl nucleoside
  modified_base 5..7: mod_base = OTHER; note = S-cEt nucleosides
  modified_base 8..9: mod_base = OTHER; note = 2'-O-methyl nucleosides
  modified_base 10..11: mod_base = OTHER; note = S-cEt nucleosides
  source 1..11: mol_type = other RNA
taagcactttg

SEQ ID NO: 17 - RNA, 10 nt, synthetic construct
  modified_base 8: mod_base = OTHER; note = thymine
  misc_feature 1..10: note = Synthetic: RG-NG-1030
  modified_base 1..2: mod_base = OTHER; note = 2'-O-methyl nucleosides
  modified_base 3..6: mod_base = OTHER; note = LNA
  modified_base 7: mod_base = OTHER; note = 2'-O-methyl nucleoside
  modified_base 8: mod_base = OTHER; note = LNA
  modified_base 10: mod_base = OTHER; note = 2'-O-methyl nucleoside
  source 1..10: mol_type = other RNA
aagcactttc

SEQ ID NO: 18 - RNA, 15 nt, synthetic construct
  modified_base 3: mod_base = OTHER; note = thymine
  modified_base 5: mod_base = OTHER; note = thymine
  misc_feature 1..15: note = Synthetic: RG-NG-1018
  modified_base 1..3: mod_base = OTHER; note = 2'-O-methoxyethyl
  modified_base 4: mod_base = OTHER; note = S-cEt nucleoside
  modified_base 5: mod_base = OTHER; note = 2'-O-methoxyethyl
  modified_base 7..8: mod_base = OTHER; note = S-cEt nucleoside
  modified_base 9: mod_base = OTHER; note = 2'-O-methyl nucleoside
  modified_base 10..12: mod_base = OTHER; note = 2'-fluoro nucleosides
  modified_base 13: mod_base = OTHER; note = 2'-O-methyl nucleoside
  modified_base 14..15: mod_base = OTHER; note = S-cEt nucleoside
  source 1..15: mol_type = other RNA
actgtaagcactttc

SEQ ID NO: 19 - RNA, 15 nt, synthetic construct
  modified_base 3: mod_base = OTHER; note = thymine
  misc_feature 1..15: note = Synthetic: RG-NG-1019
  modified_base 1..2: mod_base = OTHER; note = S-cEt nucleosides
  modified_base 4..5: mod_base = OTHER; note = S-cEt nucleosides
  modified_base 7..8: mod_base = OTHER; note = S-cEt nucleosides
  modified_base 9: mod_base = OTHER; note = 2'-O-methyl nucleoside
  modified_base 10..12: mod_base = OTHER; note = 2'-fluoro nucleosides
  modified_base 13: mod_base = OTHER; note = 2'-O-methyl nucleoside
  modified_base 14..15: mod_base = OTHER; note = S-cEt nucleosides
  source 1..15: mol_type = other RNA
actgtaagcactttc

SEQ ID NO: 20 - RNA, 13 nt, synthetic construct
  modified_base 1: mod_base = OTHER; note = thymine
  modified_base 3: mod_base = OTHER; note = thymine
  misc_feature 1..13: note = Synthetic: RG-NG-1020
  modified_base 1: mod_base = OTHER; note = 2'-O-methoxyethyl
  modified_base 2: mod_base = OTHER; note = S-cEt nucleoside
  modified_base 3: mod_base = OTHER; note = 2'-O-methoxyethyl
  modified_base 5..6: mod_base = OTHER; note = S-cEt nucleoside
  modified_base 7: mod_base = OTHER; note = 2'-O-methyl nucleoside
  modified_base 8..10: mod_base = OTHER; note = 2'-fluoro nucleosides
  modified_base 11: mod_base = OTHER; note = 2'-O-methyl nucleoside
  modified_base 12..13: mod_base = OTHER; note = S-cEt nucleoside
  source 1..13: mol_type = other RNA
tgtaagcactttc

SEQ ID NO: 21 - RNA, 11 nt, synthetic construct
  modified_base 1: mod_base = OTHER; note = thymine
  misc_feature 1..11: note = Synthetic: RG-NG-1021
  modified_base 1: mod_base = OTHER; note = 2'-O-methoxyethyl
  modified_base 3..4: mod_base = OTHER; note = S-cEt nucleoside
  modified_base 5: mod_base = OTHER; note = 2'-O-methyl nucleoside
  modified_base 6..8: mod_base = OTHER; note = 2'-fluoro nucleosides
  modified_base 9: mod_base = OTHER; note = 2'-O-methyl nucleoside
  modified_base 10..11: mod_base = OTHER; note = S-cEt nucleoside
  source 1..11: mol_type = other RNA
taagcactttc

SEQ ID NO: 22 - RNA, 11 nt, synthetic construct
  modified_base 1: mod_base = OTHER; note = thymine
  misc_feature 1..11: note = Synthetic: RG-NG-1022
  modified_base 1..2: mod_base = OTHER; note = 2'-O-methoxyethyl
  modified_base 3..4: mod_base = OTHER; note = S-cEt nucleoside
  modified_base 5: mod_base = OTHER; note = 2'-O-methyl nucleoside
  modified_base 6..8: mod_base = OTHER; note = 2'-fluoro nucleosides
  modified_base 9: mod_base = OTHER; note = 2'-O-methyl nucleoside
  modified_base 10..11: mod_base = OTHER; note = S-cEt nucleoside
  source 1..11: mol_type = other RNA
taagcactttc

SEQ ID NO: 23 - RNA, 19 nt, synthetic construct
  modified_base 2: mod_base = OTHER; note = thymine
  modified_base 7: mod_base = OTHER; note = thymine
  modified_base 9: mod_base = OTHER; note = thymine
  misc_feature 1..19: note = Synthetic: RG-NG-1040
  modified_base 1..2: mod_base = OTHER; note = 2'-O-methoxyethyl
  modified_base 3: mod_base = OTHER; note = S-cEt nucleoside
  modified_base 4..7: mod_base = OTHER; note = 2'-O-methoxyethyl
  modified_base 8: mod_base = OTHER; note = S-cEt nucleoside
  modified_base 9: mod_base = OTHER; note = 2'-O-methoxyethyl
  modified_base 10: mod_base = OTHER; note = DNA
  modified_base 11..12: mod_base = OTHER; note = S-cEt nucleoside
  modified_base 13: mod_base = OTHER; note = 2'-O-methyl nucleoside
  modified_base 14..16: mod_base = OTHER; note = 2-fluoro nucleosides
  modified_base 17: mod_base = OTHER; note = 2'-O-methyl nucleoside
  modified_base 18..19: mod_base = OTHER; note = S-cEt nucleoside
  source 1..19: mol_type = other RNA
ctgcactgtaagcactttg

SEQ ID NO: 24 - RNA, 17 nt, synthetic construct
  misc_feature 1..17: note = Synthetic: RG-NG-1032
  modified_base 1: mod_base = OTHER; note = S-cEt nucleoside
  modified_base 2..5: mod_base = OTHER; note = 2'-O-methoxyethyl
  modified_base 6: mod_base = OTHER; note = S-cEt nucleoside
  modified_base 7: mod_base = OTHER; note = 2'-O-methoxyethyl
  modified_base 8: mod_base = OTHER; note = DNA
  modified_base 9..10: mod_base = OTHER; note = S-cEt nucleoside
  modified_base 11: mod_base = OTHER; note = 2'-O-methyl nucleoside
  modified_base 12..14: mod_base = OTHER; note = 2-fluoro nucleosides
  modified_base 15: mod_base = OTHER; note = 2'-O-methyl nucleoside
  modified_base 16..17: mod_base = OTHER; note = S-cEt nucleoside
  source 1..17: mol_type = other RNA
  modified_base 5: mod_base = OTHER; note = thymine
  modified_base 7: mod_base = OTHER; note = thymine
gcactgtaagcactttg

(19) United States
(12) Patent Application Publication
(10) Pub. No.: US 2025/0136979 A1
(43) Pub. Date: May 1, 2025

(54) Methods and Compositions for Avoiding Off-Target Effects

(71) Applicant: Regulus Therapeutics Inc., San Diego, CA (US)

(72) Inventors: Denis Drygin, San Diego, CA (US); Garth A. Kinberger, San Diego, CA (US); Edmund Chun Yu Lee, San Diego, CA (US)

(73) Assignee: Regulus Therapeutics Inc., San Diego, CA (US)

(21) Appl. No.: 18/693,783

(22) PCT Filed: Oct. 7, 2022
(86) PCT No.: PCT/US2022/077767

§ 371 Date: Mar. 20, 2024

Related U.S. Application Data
(60) Provisional application No. 63/253,917, filed on Oct. 8, 2021.

(51) Int. Cl. C12N 15/113

(52) U.S. Cl. CPC C12N 15/113; C12N 2310/141; C12N 2310/315; C12N 2310/321

(57) ABSTRACT

Provided herein are compounds comprising modified oligonucleotides targeted to microRNAs, wherein the modified oligonucleotides are designed to avoid off-target effects.

### CROSS-REFERENCE TO RELATED APPLICATION

This application claims the benefit of priority of U.S. Provisional Application No. 63/253,917, filed Oct. 8, 2021, which is incorporated by reference herein in its entirety for any purpose.

### FIELD OF INVENTION

Provided herein are compositions and methods for the inhibition of microRNA activity.

### BACKGROUND

Strategies for therapeutic modulation of RNA function often employ the use of antisense oligonucleotides that are designed to bind to the RNA target through Watson-Crick base pairing, and, once bound to the target, modulate its function. Such antisense oligonucleotides are chemically modified to impart desired pharmacokinetic and pharmacodynamic properties to the oligonucleotides. Modified oligonucleotides may modulate a target RNA through a variety of mechanisms, including mechanisms that involve binding of the modified oligonucleotide to the target RNA and interference with its function without promoting degradation of the RNA (e.g., steric hindrance), as well as mechanisms that do promote degradation of the RNA after binding of the modified oligonucleotide, by activities of enzymes such as RNaseH or Argonaute 2. Numerous types of RNAs may be selected as targets of modified oligonucleotides, including messenger RNAs, pre-messenger RNAs, and non-coding RNAs such as microRNAs.

MicroRNAs (microRNAs), also known as “mature microRNA” are small (approximately 18-24 nucleotides in length), non-coding RNA molecules encoded in the genomes of plants and animals. In certain instances, highly conserved, endogenously expressed microRNAs regulate the expression of genes by binding to the 3′-untranslated regions (3′-UTR) of specific mRNAs. More than 1000 different microRNAs have been identified in plants and animals. Certain mature microRNAs appear to originate from long endogenous primary microRNA transcripts (also known as pri-microRNAs, pri-mirs, pri-miRs or pri-pre-microRNAs) that are often hundreds of nucleotides in length (Lee, et al., EMBO J., 2002, 21(17), 4663-4670).

### SUMMARY OF INVENTION

Embodiment 1. A compound comprising a modified oligonucleotide, wherein the modified oligonucleotide has the following structure in the 5′ to 3′ orientation:

(N)ₚ—(N″)—(N′)_{q}
p is from 7 to 24;
q is 0 or 1;
each N of (N)ₚ, independently, comprises a modified sugar moiety or an unmodified sugar moiety, and the nucleobase sequence of (N)ₚ is complementary to an equal-length portion of a microRNA, wherein (i) the nucleobase at position 1 of the microRNA is a uracil nucleobase and/or a cytosine nucleobase; (ii) the nucleobase at position 2 of the microRNA is a uracil nucleobase and/or a cytosine nucleobase; or (iii) the nucleobase at position 1 of the microRNA is a uracil and/or a cytosine nucleobase and the nucleobase at position 2 of the microRNA is a uracil nucleobase and/or a cytosine nucleobase;
N″ is a nucleoside comprising a modified sugar moiety or an unmodified sugar moiety, and the nucleobase of N″ is opposite position 2 of the microRNA;
wherein if q is 1, N′ is a nucleoside comprising a modified sugar moiety or an unmodified sugar moiety, and the nucleobase of N′ is opposite position 1 of the microRNA,
wherein if q is 0, the nucleobase of N″ is a uracil nucleobase, a cytosine nucleobase, or a purine nucleobase, provided that the purine nucleobase does not have a hydrogen bond acceptor at position 6,
wherein if q is 1, at least one nucleobase of N′ and N″ is a uracil nucleobase, a cytosine nucleobase, or a purine nucleobase, provided that the purine nucleobase does not have a hydrogen bond acceptor at position 6;
or a pharmaceutically acceptable salt thereof.

Embodiment 2. The compound of embodiment 1, wherein the nucleobase at position 1 of the microRNA is a uracil nucleobase, q is 1, and N′ is a uracil nucleobase, a cytosine nucleobase, or a purine nucleobase, provided that the purine nucleobase does not have a hydrogen bond acceptor at position 6.

Embodiment 3. The compound of embodiment 1 or embodiment 2, wherein the nucleobase at position 2 of the microRNA is a uracil nucleobase, and N″ is a uracil nucleobase, a cytosine nucleobase, or a purine nucleobase, provided that the purine nucleobase does not have a hydrogen bond acceptor at position 6.

Embodiment 4. The compound of embodiment 1 or embodiment 2, wherein the nucleobase at position 2 of the microRNA is a cytosine nucleobase, and N″ is a uracil nucleobase, a cytosine nucleobase, or a purine nucleobase, provided that the purine nucleobase does not have a hydrogen bond acceptor at position 6.

Embodiment 5. The compound of embodiment 3 or embodiment 4, wherein q is 0.

Embodiment 6. The compound of embodiment 1, wherein the nucleobase at position 1 of the microRNA is a cytosine nucleobase, q is 1, and N′ is a uracil nucleobase, a cytosine nucleobase, or a purine nucleobase, provided that the purine nucleobase does not have a hydrogen bond acceptor at position 6.

Embodiment 7. The compound of embodiment 1 or embodiment 6, wherein the nucleobase at position 2 of the microRNA is a cytosine nucleobase and N″ is a uracil nucleobase, a cytosine nucleobase, or a purine nucleobase, provided that the purine nucleobase does not have a hydrogen bond acceptor at position 6.

Embodiment 8. The compound of embodiment 1 or embodiment 6, wherein the nucleobase at position 2 of the microRNA is a uracil nucleobase and N″ is a uracil nucleobase, a cytosine nucleobase, or a purine nucleobase, provided that the purine nucleobase does not have a hydrogen bond acceptor at position 6.

Embodiment 9. The compound of embodiment 7 or embodiment 8, wherein q is 0.

Embodiment 10. The compound of any one of embodiments 1, 3 to 5, and 7 to 9, wherein the nucleobase of N″ is a purine nucleobase that does not have a hydrogen bond acceptor at position 6.

Embodiment 11. The compound of embodiment 10, wherein the nucleobase of N″ is selected from adenosine, 2-aminopurine, 2, 6-diaminopurine, and isoguanosine.

Embodiment 12. The compound of any one of embodiments 1 to 11, wherein the sugar moiety of N″ is not a 2′-O-methyl sugar.

Embodiment 13. The compound of any one of embodiments 1 to 11, wherein the sugar moiety of N″ is a 2′-O-methoxyethyl sugar or an S-cEt sugar.

Embodiment 14. The compound of any one of embodiments 1 to 4, 6 to 8, and 10 to 13, wherein the nucleobase of N′ is a purine nucleobase that does not have a hydrogen bond acceptor at position 6.

Embodiment 15. The compound of embodiment 14, wherein the nucleobase of N′ is selected from adenosine, 2-aminopurine, 2, 6-diaminopurine, and isoguanosine.

Embodiment 16. The compound of any one of embodiments 1 to 4, 6 to 8, and 10 to 15, wherein the sugar moiety of N′ is not a 2′-O-methyl sugar.

Embodiment 17. The compound of any one of embodiments 1 to 4, 6 to 8, and 10 to 15, wherein the sugar moiety of N′ is a 2′-O-methoxyethyl sugar or an S-cEt sugar.

Embodiment 18. The compound of any one of embodiments 1 to 17, wherein at least one internucleoside linkage is a phosphorothioate internucleoside linkage.

Embodiment 19. The compound of embodiment 18, wherein each internucleoside linkage is a phosphorothioate internucleoside linkage.

Embodiment 20. The compound of any one of embodiments 1 to 19, wherein p is 7, 8, 9, 10, 11, 12, 13, 14, 15, 16, 17, 18, 19, 20, 21, 22, 23, or 24.

Embodiment 21. The compound of any one of embodiments 1 to 20, wherein the nucleobase sequence of the modified oligonucleotide is at least 90%, is at least 95%, or is 100% complementary to the nucleobase sequence of the microRNA.

Embodiment 22. The compound of any one of embodiments 1 to 20, wherein the compound consists of the modified oligonucleotide.

Embodiment 23. The compound of any one of embodiments 1 to 22, wherein the pharmaceutically acceptable salt is a sodium salt.

Embodiment 24. A pharmaceutical composition comprising a compound of any one of embodiments 1 to 23 and a pharmaceutically acceptable diluent.

Embodiment 25. The pharmaceutical composition of embodiment 24, wherein the pharmaceutically acceptable diluent is an aqueous solution.

Embodiment 26. The pharmaceutical composition of embodiment 25, wherein the aqueous solution is a saline solution.

Embodiment 27. A pharmaceutical composition comprising a compound of any one of embodiments 1 to 23, which is a lyophilized composition.

Embodiment 28. A pharmaceutical composition consisting essentially of a compound of any one of embodiments 1 to 23 to in a saline solution.

Embodiment 29. A method for inhibiting the activity of a microRNA in a cell, comprising contacting the cell with a compound of any one of embodiments 1 to 23.

Embodiment 30. A method for inhibiting the activity of a microRNA in a subject, comprising administering to the subject a compound of any one of embodiments 1 to 23, or a pharmaceutical composition of any one of embodiments 24 to 28.

Embodiment 31. The method of embodiment 30, wherein the subject has a disease associated with the microRNA.

Embodiment 32. The method of embodiment 30 or 31, comprising administering a therapeutically effective amount of the compound.

Embodiment 33. The method of any one of embodiments 30 to 32, wherein the subject is a human subject.

Embodiment 34. A compound of any one of embodiments 1 to 23, or a pharmaceutical composition of any one of embodiments 27 to 28, for use in therapy.

### BRIEF DESCRIPTION OF FIGURES

FIG. 1. Purine nucleobase structures.

FIG. 2A-2C. Efficacy of RG-NG-1015 in the Pkd2-KO model of PKD. Effects of treatment on (A) kidney-to-body weight ratio, (B) blood urea nitrogen (BUN) level, and (C) blood creatinine level.

FIG. 3. Maximum Tolerated Dose (MTD) study and Comparative Dose Assessment of RG-NG-1001, RGLS4326, and RG-NG-1017. 6-7-week-old male C57BL/6J mice were dosed with a single intracerebroventricular (ICV) injection of RG-NG-1001 and RGLS4326 (anti-miR-17 oligos that inhibit AMPA-R) and RG-NG-1017 (anti-miR-17 oligos that does not inhibit AMPA-R; RG-NG-1017) at different dose levels in 4 μL volume and monitored for 7 days. Mortality of the mice is indicated for the three different compounds at different dosages.

### DETAILED DESCRIPTION

Unless defined otherwise, all technical and scientific terms used herein have the same meaning as is commonly understood by one of skill in the arts to which the invention belongs. Unless specific definitions are provided, the nomenclature utilized in connection with, and the procedures and techniques of, analytical chemistry, synthetic organic chemistry, and medicinal and pharmaceutical chemistry described herein are those well-known and commonly used in the art. In the event that there is a plurality of definitions for terms herein, those in this section prevail. Standard techniques may be used for chemical synthesis, chemical analysis, pharmaceutical preparation, formulation and delivery, and treatment of subjects. Certain such techniques and procedures may be found for example in “Carbohydrate Modifications in Antisense Research” Edited by Sanghvi and Cook, American Chemical Society, Washington D.C., 1994; and “Remington's Pharmaceutical Sciences,” Mack Publishing Co., Easton, Pa., 18th edition, 1990; and which is hereby incorporated by reference for any purpose. Where permitted, all patents, patent applications, published applications and publications, GENBANK sequences, websites and other published materials referred to throughout the entire disclosure herein, unless noted otherwise, are incorporated by reference in their entirety. Where reference is made to a URL or other such identifier or address, it is understood that such identifiers can change and particular information on the internet can change, but equivalent information can be found by searching the internet. Reference thereto evidences the availability and public dissemination of such information.

Before the present compositions and methods are disclosed and described, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

### Definitions

“Subject” means a human or non-human animal selected for treatment or therapy.

“Subject in need thereof” means a subject that is identified as in need of a therapy or treatment.

“Subject suspected of having” means a subject exhibiting one or more clinical indicators of a disease.

“Administering” means providing a pharmaceutical agent or composition to a subject, and includes, but is not limited to, administering by a medical professional and self-administering.

“Parenteral administration” means administration through injection or infusion.

Parenteral administration includes, but is not limited to, subcutaneous administration, intravenous administration, and intramuscular administration.

“Subcutaneous administration” means administration just below the skin.

“Intravenous administration” means administration into a vein.

“Administered concomitantly” refers to the co-administration of two or more agents in any manner in which the pharmacological effects of both are manifest in the patient at the same time. Concomitant administration does not require that both agents be administered in a single pharmaceutical composition, in the same dosage form, or by the same route of administration. The effects of both agents need not manifest themselves at the same time. The effects need only be overlapping for a period and need not be coextensive.

“Duration” means the period during which an activity or event continues. In certain embodiments, the duration of treatment is the period during which doses of a pharmaceutical agent or pharmaceutical composition are administered.

“Therapy” means a disease treatment method. In certain embodiments, therapy includes, but is not limited to, administration of one or more pharmaceutical agents to a subject having a disease.

“Treat” means to apply one or more specific procedures used for the amelioration of at least one indicator of a disease. In certain embodiments, the specific procedure is the administration of one or more pharmaceutical agents. In certain embodiments, treatment of PKD includes, but is not limited to, reducing total kidney volume, improving kidney function, reducing hypertension, and/or reducing kidney pain.

“Ameliorate” means to lessen the severity of at least one indicator of a condition or disease. In certain embodiments, amelioration includes a delay or slowing in the progression of one or more indicators of a condition or disease. The severity of indicators may be determined by subjective or objective measures which are known to those skilled in the art.

“At risk for developing” means the state in which a subject is predisposed to developing a condition or disease. In certain embodiments, a subject at risk for developing a condition or disease exhibits one or more symptoms of the condition or disease, but does not exhibit a sufficient number of symptoms to be diagnosed with the condition or disease. In certain embodiments, a subject at risk for developing a condition or disease exhibits one or more symptoms of the condition or disease, but to a lesser extent required to be diagnosed with the condition or disease.

“Prevent the onset of” means to prevent the development of a condition or disease in a subject who is at risk for developing the disease or condition. In certain embodiments, a subject at risk for developing the disease or condition receives treatment similar to the treatment received by a subject who already has the disease or condition.

“Delay the onset of” means to delay the development of a condition or disease in a subject who is at risk for developing the disease or condition. In certain embodiments, a subject at risk for developing the disease or condition receives treatment similar to the treatment received by a subject who already has the disease or condition.

“Dose” means a specified quantity of a pharmaceutical agent provided in a single administration. In certain embodiments, a dose may be administered in two or more boluses, tablets, or injections. For example, in certain embodiments, where subcutaneous administration is desired, the desired dose requires a volume not easily accommodated by a single injection. In such embodiments, two or more injections may be used to achieve the desired dose. In certain embodiments, a dose may be administered in two or more injections to minimize injection site reaction in an individual. In certain embodiments, a dose is administered as a slow infusion.

“Dosage unit” means a form in which a pharmaceutical agent is provided. In certain embodiments, a dosage unit is a vial containing lyophilized oligonucleotide. In certain embodiments, a dosage unit is a vial containing reconstituted oligonucleotide.

“Therapeutically effective amount” refers to an amount of a pharmaceutical agent that provides a therapeutic benefit to a subject.

“Pharmaceutical composition” means a mixture of substances suitable for administering to an individual that includes a pharmaceutical agent. For example, a pharmaceutical composition may comprise a sterile aqueous solution.

“Pharmaceutical agent” means a substance that provides a therapeutic effect when administered to a subject.

“Active pharmaceutical ingredient” means the substance in a pharmaceutical composition that provides a desired effect.

“Pharmaceutically acceptable salt” means a physiologically and pharmaceutically acceptable salt of a compound provided herein, i.e., a salt that retains the desired biological activity of the compound and does not have undesired toxicological effects when administered to a subject. Nonlimiting exemplary pharmaceutically acceptable salts of compounds provided herein include sodium and potassium salt forms. The terms “compound,” “oligonucleotide,” and “modified oligonucleotide” as used herein include pharmaceutically acceptable salts thereof unless specifically indicated otherwise.

“Saline solution” means a solution of sodium chloride in water.

“Improved organ function” means a change in organ function toward normal limits. In certain embodiments, organ function is assessed by measuring molecules found in a subject's blood or urine. For example, in certain embodiments, improved kidney function is measured by a reduction in blood urea nitrogen level, a reduction in proteinuria, a reduction in albuminuria, etc.

“Acceptable safety profile” means a pattern of side effects that is within clinically acceptable limits.

“Side effect” means a physiological response attributable to a treatment other than desired effects. In certain embodiments, side effects include, without limitation, injection site reactions, liver function test abnormalities, kidney function abnormalities, liver toxicity, renal toxicity, central nervous system abnormalities, and myopathies. Such side effects may be detected directly or indirectly. For example, increased aminotransferase levels in serum may indicate liver toxicity or liver function abnormality. For example, increased bilirubin may indicate liver toxicity or liver function abnormality.

The term “blood” as used herein, encompasses whole blood and blood fractions, such as serum and plasma.

“Anti-miR” means an oligonucleotide having a nucleobase sequence complementary to a microRNA. In certain embodiments, an anti-miR is a modified oligonucleotide.

“Seed sequence” means nucleobases 2 through 7 of a microRNA, counting from the 5′ end of the microRNA.

“Target nucleic acid” means a nucleic acid to which an oligomeric compound is designed to hybridize.

“Targeting” means the process of design and selection of nucleobase sequence that will hybridize to a target nucleic acid.

“Targeted to” means having a nucleobase sequence that will allow hybridization to a target nucleic acid.

“Modulation” means a perturbation of function, amount, or activity. In certain embodiments, modulation means an increase in function, amount, or activity. In certain embodiments, modulation means a decrease in function, amount, or activity.

“Expression” means any functions and steps by which a gene's coded information is converted into structures present and operating in a cell.

“Nucleobase sequence” means the order of contiguous nucleobases in an oligomeric compound or nucleic acid, typically listed in a 5′ to 3′ orientation, and independent of any sugar, linkage, and/or nucleobase modification.

“Contiguous nucleobases” means nucleobases immediately adjacent to each other in a nucleic acid.

“Nucleobase complementarity” means the ability of two nucleobases to pair non-covalently via hydrogen bonding.

“Complementary” means that one nucleic acid is capable of hybridizing to another nucleic acid or oligonucleotide. In certain embodiments, complementary refers to an oligonucleotide capable of hybridizing to a target nucleic acid.

“Fully complementary” means each nucleobase of an oligonucleotide is capable of pairing with a nucleobase at each corresponding position in a target nucleic acid. In certain embodiments, an oligonucleotide is fully complementary (also referred to as 100% complementary) to a microRNA, i.e. each nucleobase of the oligonucleotide is complementary to a nucleobase at a corresponding position in the microRNA. A modified oligonucleotide may be fully complementary to a microRNA, and have a number of linked nucleosides that is less than the length of the microRNA. For example, an oligonucleotide with 16 linked nucleosides, where each nucleobase of the oligonucleotide is complementary to a nucleobase at a corresponding position in a microRNA, is fully complementary to the microRNA. In certain embodiments, an oligonucleotide wherein each nucleobase has complementarity to a nucleobase within a region of a microRNA stem-loop sequence is fully complementary to the microRNA stem-loop sequence.

“Percent complementarity” means the percentage of nucleobases of an oligonucleotide that are complementary to an equal-length portion of a target nucleic acid. Percent complementarity is calculated by dividing the number of nucleobases of the oligonucleotide that are complementary to nucleobases at corresponding positions in the target nucleic acid by the total number of nucleobases in the oligonucleotide.

“Percent identity” means the number of nucleobases in a first nucleic acid that are identical to nucleobases at corresponding positions in a second nucleic acid, divided by the total number of nucleobases in the first nucleic acid. In certain embodiments, the first nucleic acid is a microRNA and the second nucleic acid is a microRNA. In certain embodiments, the first nucleic acid is an oligonucleotide and the second nucleic acid is an oligonucleotide.

“Hybridize” means the annealing of complementary nucleic acids that occurs through nucleobase complementarity.

“Mismatch” means a nucleobase of a first nucleic acid that is not capable of Watson-Crick pairing with a nucleobase at a corresponding position of a second nucleic acid.

“Identical” in the context of nucleobase sequences, means having the same nucleobase sequence, independent of sugar, linkage, and/or nucleobase modifications and independent of the methylation state of any pyrimidines present.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (microma.sanger.ac.uk/). In certain embodiments, microRNA is abbreviated as “miR.”

“microRNA-regulated transcript” means a transcript that is regulated by a microRNA.

“Seed match sequence” means a nucleobase sequence that is complementary to a seed sequence, and is the same length as the seed sequence.

“Oligomeric compound” means a compound that comprises a plurality of linked monomeric subunits. Oligomeric compounds include oligonucleotides.

“Oligonucleotide” means a compound comprising a plurality of linked nucleosides, each of which can be modified or unmodified, independent from one another.

“Naturally occurring internucleoside linkage” means a 3′ to 5′ phosphodiester linkage between nucleosides.

“Natural sugar” means a sugar found in DNA (2′-H) or RNA (2′-OH).

“Internucleoside linkage” means a covalent linkage between adjacent nucleosides.

“Linked nucleosides” means nucleosides joined by a covalent linkage.

“Nucleobase” means a heterocyclic moiety capable of non-covalently pairing with another nucleobase.

“Nucleoside” means a nucleobase linked to a sugar moiety.

“Nucleotide” means a nucleoside having a phosphate group covalently linked to the sugar portion of a nucleoside.

“Compound comprising a modified oligonucleotide consisting of” a number of linked nucleosides means a compound that includes a modified oligonucleotide having the specified number of linked nucleosides. Thus, the compound may include additional substituents or conjugates. Unless otherwise indicated, the modified oligonucleotide is not hybridized to a complementary strand and the compound does not include any additional nucleosides beyond those of the modified oligonucleotide.

“Modified oligonucleotide” means a single-stranded oligonucleotide having one or more modifications relative to a naturally occurring terminus, sugar, nucleobase, and/or internucleoside linkage. A modified oligonucleotide may comprise unmodified nucleosides.

“Modified nucleoside” means a nucleoside having any change from a naturally occurring nucleoside. A modified nucleoside may have a modified sugar and an unmodified nucleobase. A modified nucleoside may have a modified sugar and a modified nucleobase. A modified nucleoside may have a natural sugar and a modified nucleobase. In certain embodiments, a modified nucleoside is a bicyclic nucleoside. In certain embodiments, a modified nucleoside is a non-bicyclic nucleoside.

“Modified internucleoside linkage” means any change from a naturally occurring internucleoside linkage.

“Phosphorothioate internucleoside linkage” means a linkage between nucleosides where one of the non-bridging atoms is a sulfur atom.

“Modified sugar moiety” means substitution and/or any change from a natural sugar.

“Unmodified nucleobase” means the naturally occurring heterocyclic bases of RNA or DNA: the purine bases adenine (A) and guanine (G), and the pyrimidine bases thymine (T), cytosine (C) (including 5-methylcytosine), and uracil (U).

“5-methylcytosine” means a cytosine comprising a methyl group attached to the 5 position.

“Non-methylated cytosine” means a cytosine that does not have a methyl group attached to the 5 position.

“Modified nucleobase” means any nucleobase that is not an unmodified nucleobase.

“Sugar moiety” means a naturally occurring furanosyl or a modified sugar moiety.

“Modified sugar moiety” means a substituted sugar moiety or a sugar surrogate.

“2′-O-methyl sugar” or “2′-OMe sugar” means a sugar having an O-methyl modification at the 2′ position.

“2′-O-methoxyethyl sugar” or “2′-MOE sugar” means a sugar having an O-methoxyethyl modification at the 2′ position.

“2′-fluoro” or “2′-F” means a sugar having a fluoro modification of the 2′ position.

“Bicyclic sugar moiety” means a modified sugar moiety comprising a 4 to 7 membered ring (including by not limited to a furanosyl) comprising a bridge connecting two atoms of the 4 to 7 membered ring to form a second ring, resulting in a bicyclic structure. In certain embodiments, the 4 to 7 membered ring is a sugar ring. In certain embodiments, the 4 to 7 membered ring is a furanosyl. In certain such embodiments, the bridge connects the 2′-carbon and the 4′-carbon of the furanosyl. Nonlimiting exemplary bicyclic sugar moieties include LNA, ENA, cEt, S-cEt, and R-cEt.

“Locked nucleic acid (LNA) sugar moiety” means a substituted sugar moiety comprising a (CH₂)—O bridge between the 4′ and 2′ furanose ring atoms.

“ENA sugar moiety” means a substituted sugar moiety comprising a (CH₂)₂-O bridge between the 4′ and 2′ furanose ring atoms.

“Constrained ethyl (cEt) sugar moiety” means a substituted sugar moiety comprising a CH(CH₃)—O bridge between the 4′ and the 2′ furanose ring atoms. In certain embodiments, the CH(CH₃)—O bridge is constrained in the S orientation. In certain embodiments, the CH(CH₃)—O is constrained in the R orientation.

“S-cEt sugar moiety” means a substituted sugar moiety comprising an S-constrained CH(CH₃)—O bridge between the 4′ and the 2′ furanose ring atoms.

“R-cEt sugar moiety” means a substituted sugar moiety comprising an R-constrained CH(CH₃)—O bridge between the 4′ and the 2′ furanose ring atoms.

“2′-O-methyl nucleoside” means a 2′-modified nucleoside having a 2′-O-methyl sugar modification.

“2′-O-methoxyethyl nucleoside” means a 2′-modified nucleoside having a 2′-O-methoxyethyl sugar modification. A 2′-O-methoxyethyl nucleoside may comprise a modified or unmodified nucleobase.

“2′-fluoro nucleoside” means a 2′-modified nucleoside having a 2′-fluoro sugar modification. A 2′-fluoro nucleoside may comprise a modified or unmodified nucleobase.

“Bicyclic nucleoside” means a 2′-modified nucleoside having a bicyclic sugar moiety. A bicyclic nucleoside may have a modified or unmodified nucleobase.

“cEt nucleoside” means a nucleoside comprising a cEt sugar moiety. A cEt nucleoside may comprise a modified or unmodified nucleobase.

“S-cEt nucleoside” means a nucleoside comprising an S-cEt sugar moiety.

“R-cEt nucleoside” means a nucleoside comprising an R-cEt sugar moiety.

“β-D-deoxyribonucleoside” means a naturally occurring DNA nucleoside.

“β-D-ribonucleoside” means a naturally occurring RNA nucleoside.

“LNA nucleoside” means a nucleoside comprising a LNA sugar moiety.

“ENA nucleoside” means a nucleoside comprising an ENA sugar moiety.

“Hydrogen bond acceptor” means the component of a hydrogen bond that does not supply the shared hydrogen atom.

“Hydrogen bond donor” means the bond or molecule that supplies the hydrogen atom of a hydrogen bond.

### Overview

The anti-miR-17 compound RGLS4326 was discovered by screening a chemically diverse and rationally designed library of anti-miR-17 oligonucleotides for optimal pharmaceutical properties. RGLS4326 preferentially distributes to kidney and collecting duct-derived cysts, displaces miR-17 from translationally active polysomes, and de-represses multiple miR-17 mRNA targets including Pkd1 and Pkd2. Importantly, RGLS4326 attenuates cyst growth in human in vitro ADPKD models and multiple PKD mouse models after subcutaneous administration. A phase 1b clinical trial of RGLS4326 for the treatment of patients with autosomal dominant polycystic kidney disease (ADPKD) was initiated in October 2020.

Subsequent to the initiation of the phase 1b clinical trial, nonclinical toxicology studies revealed CNS-related findings, including abnormal gait, reduced motor activity, and/or prostration, at high doses of RGLS4326 in mice. RGLS4326 was found to be an antagonist of the AMPA receptor (AMPAR), a glutamate receptor and ion channel on excitatory synapses in the central nervous system (CNS) that mediates fast excitatory neurotransmission and, therefore, is a key component of all neuronal networks. Antagonism of the AMPA receptor could explain the CNS-mediated findings observed at high doses of RGLS4326 in nonclinical toxicology models. While no such CNS-related findings were observed in human subjects, it is nonetheless preferable to avoid antagonism of the AMPA receptor. Accordingly, a library of anti-miR-17 compounds was screened to identify compounds with physicochemical and pharmacological properties comparable to RGLS4326, that do not antagonize the AMPA receptor. A particular anti-miR structure was identified, that inhibits microRNA activity and also avoids the off-target effect of antagonism of the AMPA receptor. Such structures and their uses to inhibit microRNAs are provided herein.

### Compounds

Modified oligonucleotides designed to be complementary to a microRNA may comprise a guanosine nucleobase at a position complementary to a cytosine nucleobase of the microRNA. The guanosine nucleobase pairs with the cytosine nucleobase via canonical Watson-Crick base pairing, as shown in the following structure:

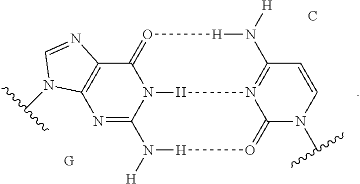

A guanosine nucleobase may also pair with a uracil nucleobase through a G-U wobble base pairing, as shown in the following structure:

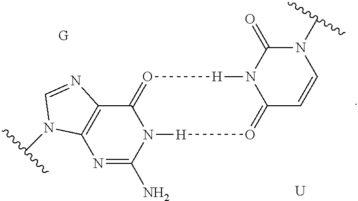

A G-U wobble base pair has a thermodynamic stability similar to that of a G-C Watson-Crick base pair. Accordingly, a modified oligonucleotide designed to be complementary to a microRNA may comprise a guanosine nucleobase positioned to be complementary to a uracil nucleobase.

Numerous microRNA sequences comprise a uracil or cytosine nucleobase at the first and/or second position of the microRNA (counting from the 5′ terminus). Modified oligonucleotide complementary to these microRNAs may comprise a guanosine nucleobase at a position complementary to the first and/or second position of the microRNA. As demonstrated herein, a guanosine at the 3′ terminus of a modified oligonucleotide complementary to a microRNA may result in the off-target effect of antagonizing the AMPA receptor, which may in turn lead to undesirable side effects in vivo. Accordingly, provided herein is a modified oligonucleotide structure designed to avoid antagonism of the AMPA receptor.

The atoms of purine nucleobases are numbered one through nine, according to standard numbering convention for nucleobases, as shown in the following structure:

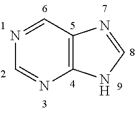

Atoms or groups bonded to the nucleobase ring atom have the same number as the ring atom to which they are bonded.

Certain nucleobases, for example guanosine and inosine, contain hydrogen-bond acceptors at position 6. The hydrogen-bond acceptor at position 6 of guanosine is the oxygen bonded to the position 6 nitrogen. The hydrogen-bond acceptor at position 6 of inosine is the oxygen bonded to the position 6 nitrogen.

Purine nucleobases that do not have a hydrogen-bond acceptor at position 6 include, without limitation, 2-aminopurine, 2,6-diaminopurine, isoguanosine, and adenosine. The NH₂ present at position 6 of each of 2,6-diaminopurine, isoguanosine, and adenosine functions as a hydrogen-bond donor. The position 6 nitrogen of 2-aminopurine also functions as a hydrogen-bond donor.

A compound comprising a modified oligonucleotide, wherein the modified oligonucleotide has the following structure in the 5′ to 3′ orientation:

(N)ₚ—(N″)—(N′)_{q},

where p is from 7 to 24; q is 0 or 1; each N of (N)ₚ, independently, comprises a modified sugar moiety or an unmodified sugar moiety, and the nucleobase sequence of (N)ₚ is complementary to an equal-length portion of a microRNA, wherein (i) the nucleobase at position 1 of the microRNA is a uracil nucleobase and/or a cytosine nucleobase; (ii) the nucleobase at position 2 of the microRNA is a uracil nucleobase and/or a cytosine nucleobase; or (iii) the nucleobase at position 1 of the microRNA is a uracil and/or a cytosine nucleobase and the nucleobase at position 2 of the microRNA is a uracil nucleobase and/or a cytosine nucleobase; N″ is a nucleoside comprising a modified sugar moiety or an unmodified sugar moiety, and the nucleobase of N″ is opposite position 2 of the microRNA; wherein if q is 1, N′ is a nucleoside comprising a modified sugar moiety or an unmodified sugar moiety, and the nucleobase of N′ is opposite position 1 of the microRNA, wherein if q is 0, the nucleobase of N″ is a uracil nucleobase, a cytosine nucleobase, or a purine nucleobase, provided that the purine nucleobase does not have a hydrogen bond acceptor at position 6, wherein if q is 1, at least one nucleobase of N′ and N″ is a uracil nucleobase, a cytosine nucleobase, or a purine nucleobase, provided that the purine nucleobase does not have a hydrogen bond acceptor at position 6; or a pharmaceutically acceptable salt thereof.

In some embodiments, the nucleobase at position 1 of the microRNA is a uracil nucleobase, q is 1, and N′ is a uracil nucleobase, a cytosine nucleobase, or a purine nucleobase, provided that the purine nucleobase does not have a hydrogen bond acceptor at position 6.

In some embodiments, the nucleobase at position 2 of the microRNA is a uracil nucleobase, and N″ is a uracil nucleobase, a cytosine nucleobase, or a purine nucleobase, provided that the purine nucleobase does not have a hydrogen bond acceptor at position 6.

In some embodiments, the nucleobase at position 2 of the microRNA is a cytosine nucleobase, and N″ is a uracil nucleobase, a cytosine nucleobase, or a purine nucleobase, provided that the purine nucleobase does not have a hydrogen bond acceptor at position 6.

In some embodiments, the nucleobase at position 1 of the microRNA is a cytosine nucleobase, q is 1, and N′ is a uracil nucleobase, a cytosine nucleobase, or a purine nucleobase, provided that the purine nucleobase does not have a hydrogen bond acceptor at position 6.

In some embodiments, the nucleobase at position 2 of the microRNA is a cytosine nucleobase and N″ is a uracil nucleobase, a cytosine nucleobase, or a purine nucleobase, provided that the purine nucleobase does not have a hydrogen bond acceptor at position 6.

In some embodiments, the nucleobase at position 2 of the microRNA is a uracil nucleobase and N″ is a uracil nucleobase, a cytosine nucleobase, or a purine nucleobase, provided that the purine nucleobase does not have a hydrogen bond acceptor at position 6.

In some embodiments, q is 0.

In some embodiments, the nucleobase of N″ is a purine nucleobase that does not have a hydrogen bond acceptor at position 6.

In some embodiments, the nucleobase of N″ is selected from adenosine, 2-aminopurine, 2, 6-diaminopurine, and isoguanosine.

In some embodiments, the sugar moiety of N″ is not a 2′-O-methyl sugar.

In some embodiments, the sugar moiety of N″ is a 2′-O-methoxyethyl sugar or an S-cEt sugar.

In some embodiments, the nucleobase of N′ is a purine nucleobase that does not have a hydrogen bond acceptor at position 6.

In some embodiments, the nucleobase of N′ is selected from adenosine, 2-aminopurine, 2, 6-diaminopurine, and isoguanosine.

In some embodiments, the sugar moiety of N′ is not a 2′-O-methyl sugar.

In some embodiments, the sugar moiety of N′ is a 2′-O-methoxyethyl sugar or an S-cEt sugar.

In some embodiments, at least one internucleoside linkage is a phosphorothioate internucleoside linkage. In some embodiments, each internucleoside linkage is a phosphorothioate internucleoside linkage.

In some embodiments, p is 7, 8, 9, 10, 11, 12, 13, 14, 15, 16, 17, 18, 19, 20, 21, 22, 23, or 24.

In some embodiments, the nucleobase sequence of the modified oligonucleotide is at least 90%, is at least 95%, or is 100% complementary to the nucleobase sequence of the microRNA.

In some embodiments, the compound consists of the modified oligonucleotide.

In some embodiments, the pharmaceutically acceptable salt is a sodium salt.

In some embodiments, a pharmaceutical composition consists essentially of a compound provided herein.

In some embodiments, a pharmaceutically acceptable salt of a modified oligonucleotide comprises fewer cationic counterions (such as Na⁺) than there are phosphorothioate and/or phosphodiester linkages per molecule (i.e., some phosphorothioate and/or phosphodiester linkages are protonated). In some embodiments, a pharmaceutically acceptable salt of a modified oligonucleotide having the length of N nucleotides comprises fewer than N cationic counterions (such as Na) per molecule, with the remaining linkages being protonated.

### Certain Uses of the Invention

Provided herein are methods for inhibiting the activity of one or more microRNAs in a cell, comprising contacting a cell with a compound provided herein, which comprises a nucleobase sequence complementary to the microRNA.

Provided herein are methods for inhibiting the activity of one or more microRNAs in a subject, comprising administering to the subject a compound or a pharmaceutical composition provided herein. In certain embodiments, the subject has a disease associated with one or more microRNAs. In some embodiments, a therapeutically effective amount of the compound is administered to the subject.

In any of the embodiments provided herein, the subject is a human subject.

Any of the compounds described herein may be for use in therapy.

Any of the modified oligonucleotides described herein may be for use in therapy.

Any of the compounds provided herein may be for use in the preparation of a medicament.

Any of the modified oligonucleotides provided herein may be for use in the preparation of a medicament.

Any of the pharmaceutical compositions provided herein may be for use in therapy.

### Certain Additional Therapies

Treatments for polycystic kidney disease or any of the conditions listed herein may comprise more than one therapy.

In certain embodiments, the at least one additional therapy comprises a pharmaceutical agent.

In certain embodiments, pharmaceutical agents include anti-inflammatory agents. In certain embodiments, an anti-inflammatory agent is a steroidal anti-inflammatory agent. In certain embodiments, a steroid anti-inflammatory agent is a corticosteroid. In certain embodiments, a corticosteroid is prednisone. In certain embodiments, an anti-inflammatory agent is a non-steroidal anti-inflammatory drug. In certain embodiments, a non-steroidal anti-inflammatory agent is ibuprofen, a COX—I inhibitor, or a COX-2 inhibitor.

In certain embodiments, a pharmaceutical agent is a pharmaceutical agent that blocks one or more responses to fibrogenic signals.

In certain embodiments, an additional therapy may be a pharmaceutical agent that enhances the body's immune system, including low-dose cyclophosphamide, thymostimulin, vitamins and nutritional supplements (e.g., antioxidants, including vitamins A, C, E, beta-carotene, zinc, selenium, glutathione, coenzyme Q-10 and echinacea), and vaccines, e.g., the immunostimulating complex (ISCOM), which comprises a vaccine formulation that combines a multimeric presentation of antigen and an adjuvant.

In certain embodiments, the additional therapy is selected to treat or ameliorate a side effect of one or more pharmaceutical compositions of the present invention. Such side effects include, without limitation, injection site reactions, liver function test abnormalities, kidney function abnormalities, liver toxicity, renal toxicity, central nervous system abnormalities, and myopathies. For example, increased aminotransferase levels in serum may indicate liver toxicity or liver function abnormality. For example, increased bilirubin may indicate liver toxicity or liver function abnormality.

### Certain MicroRNA Nucleobase Sequences

In certain embodiments, a modified oligonucleotide comprises a nucleobase sequence that is complementary to a seed sequence, i.e. a modified oligonucleotide comprises a seed-match sequence complementary to positions 2 through 7 of the microRNA.

In certain embodiments, a modified oligonucleotide consists of 8 to 26 linked nucleosides. In certain embodiments, a modified oligonucleotide consists of 8 to 12 linked nucleosides. In certain embodiments, a modified oligonucleotide consists of 12 to 26 linked nucleosides. In certain embodiments, a modified oligonucleotide consists of 15 to 26 linked nucleosides. In certain embodiments, a modified oligonucleotide consists of 15 to 19 linked nucleosides. In certain embodiments, a modified oligonucleotide consists of 15 to 16 linked nucleosides. In certain embodiments, a modified oligonucleotide consists of 17 to 23 linked nucleosides. In certain embodiments, a modified oligonucleotide consists of 19 to 23 linked nucleosides.

In certain embodiments, a modified oligonucleotide consists of 8 linked nucleosides. In certain embodiments, a modified oligonucleotide consists of 9 linked nucleosides. In certain embodiments, a modified oligonucleotide consists of 10 linked nucleosides. In certain embodiments, a modified oligonucleotide consists of 11 linked nucleosides. In certain embodiments, a modified oligonucleotide consists of 12 linked nucleosides. In certain embodiments, a modified oligonucleotide consists of 13 linked nucleosides. In certain embodiments, a modified oligonucleotide consists of 14 linked nucleosides. In certain embodiments, a modified oligonucleotide consists of 15 linked nucleosides. In certain embodiments, a modified oligonucleotide consists of 16 linked nucleosides. In certain embodiments, a modified oligonucleotide consists of 17 linked nucleosides. In certain embodiments, a modified oligonucleotide consists of 18 linked nucleosides. In certain embodiments, a modified oligonucleotide consists of 19 linked nucleosides. In certain embodiments, a modified oligonucleotide consists of 20 linked nucleosides. In certain embodiments, a modified oligonucleotide consists of 21 linked nucleosides. In certain embodiments, a modified oligonucleotide consists of 22 linked nucleosides. In certain embodiments, a modified oligonucleotide consists of 23 linked nucleosides. In certain embodiments, a modified oligonucleotide consists of 24 linked nucleosides. In certain embodiments, a modified oligonucleotide consists of 25 linked nucleosides. In certain embodiments, a modified oligonucleotide consists of 26 linked nucleosides.

In certain embodiments, a modified oligonucleotide comprises one or more 5-methylcytosines. In certain embodiments, each cytosine of a modified oligonucleotide comprises a 5-methylcytosine.

In certain embodiments, the number of linked nucleosides of a modified oligonucleotide is less than the length of its target microRNA. A modified oligonucleotide having a number of linked nucleosides that is less than the length of the target microRNA, wherein each nucleobase of the modified oligonucleotide is complementary to a nucleobase at a corresponding position of the target microRNA, is considered to be a modified oligonucleotide having a nucleobase sequence that is fully complementary (also referred to as 100% complementary) to a region of the target microRNA sequence. For example, a modified oligonucleotide consisting of 9 linked nucleosides, where each nucleobase is complementary to a corresponding position of the microRNA, is fully complementary to the microRNA.

In certain embodiments, a modified oligonucleotide has a nucleobase sequence having one mismatch with respect to the nucleobase sequence of a target microRNA. In certain embodiments, a modified oligonucleotide has a nucleobase sequence having two mismatches with respect to the nucleobase sequence of a target microRNA. In certain such embodiments, a modified oligonucleotide has a nucleobase sequence having no more than two mismatches with respect to the nucleobase sequence of a target microRNA. In certain such embodiments, the mismatched nucleobases are contiguous. In certain such embodiments, the mismatched nucleobases are not contiguous.

Although the sequence listing accompanying this filing identifies each nucleobase sequence as either “RNA” or “DNA” as required, in practice, those sequences may be modified with a combination of chemical modifications specified herein. One of skill in the art will readily appreciate that in the sequence listing, such designation as “RNA” or “DNA” to describe modified oligonucleotides is somewhat arbitrary. For example, a modified oligonucleotide provided herein comprising a nucleoside comprising a 2′-O-methoxyethyl sugar moiety and a thymine base may described as a DNA residue in the sequence listing, even though the nucleoside is modified and is not a natural DNA nucleoside.

Accordingly, nucleic acid sequences provided in the sequence listing are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” in the sequence listing encompasses any oligonucleotide having such nucleobase sequence, whether modified or unmodified, including, but not limited to, such compounds comprising RNA bases, such as those having sequence “AUCGAUCG” and those having some DNA bases and some RNA bases such as “AUCGATCG” and oligonucleotides having other modified bases, such as “AT^{me}CGAUCG,” wherein ^{me}C indicates a 5-methylcytosine.

### Certain Modifications

In certain embodiments, oligonucleotides provided herein may comprise one or more modifications to a nucleobase, sugar, and/or internucleoside linkage, and as such is a modified oligonucleotide. A modified nucleobase, sugar, and/or internucleoside linkage may be selected over an unmodified form because of desirable properties such as, for example, enhanced cellular uptake, enhanced affinity for other oligonucleotides or nucleic acid targets and increased stability in the presence of nucleases.

In certain embodiments, a modified oligonucleotide comprises one or more modified nucleosides.

In certain embodiments, a modified nucleoside is a sugar-modified nucleoside. In certain such embodiments, the sugar-modified nucleosides may further comprise a natural or modified heterocyclic base moiety and/or may be connected to another nucleoside through a natural or modified internucleoside linkage and/or may include further modifications independent from the sugar modification. In certain embodiments, a sugar modified nucleoside is a 2′-modified nucleoside, wherein the sugar ring is modified at the 2′ carbon from natural ribose or 2′-deoxy-ribose.

In certain embodiments, a 2′-modified nucleoside has a bicyclic sugar moiety. In certain such embodiments, the bicyclic sugar moiety is a D sugar in the alpha configuration. In certain such embodiments, the bicyclic sugar moiety is a D sugar in the beta configuration. In certain such embodiments, the bicyclic sugar moiety is an L sugar in the alpha configuration. In certain such embodiments, the bicyclic sugar moiety is an L sugar in the beta configuration.

Nucleosides comprising such bicyclic sugar moieties are referred to as bicyclic nucleosides or BNAs. In certain embodiments, bicyclic nucleosides include, but are not limited to, (A) α-L-methyleneoxy (4′-CH₂—O-2′) BNA; (B) β-D-methyleneoxy (4′-CH₂—O-2′) BNA; (C) ethyleneoxy (4′-(CH₂)₂-O-2′) BNA; (D) aminooxy (4′-CH₂—O—N(R)-2′) BNA; (E) oxyamino (4′-CH₂—N(R)—O-2′) BNA; (F) methyl(methyleneoxy) (4′-CH(CH₃)—O-2′) BNA (also referred to as constrained ethyl or cEt); (G) methylene-thio (4′-CH₂—S-2′) BNA; (H) methylene-amino (4′-CH2-N(R)-2′) BNA; (I) methyl carbocyclic (4′-CH₂—CH(CH₃)-2′) BNA; (J) c-MOE (4′-CH(CH₂—OMe)-O-2′) BNA and (K) propylene carbocyclic (4′-(CH₂)₃-2′) BNA as depicted below.

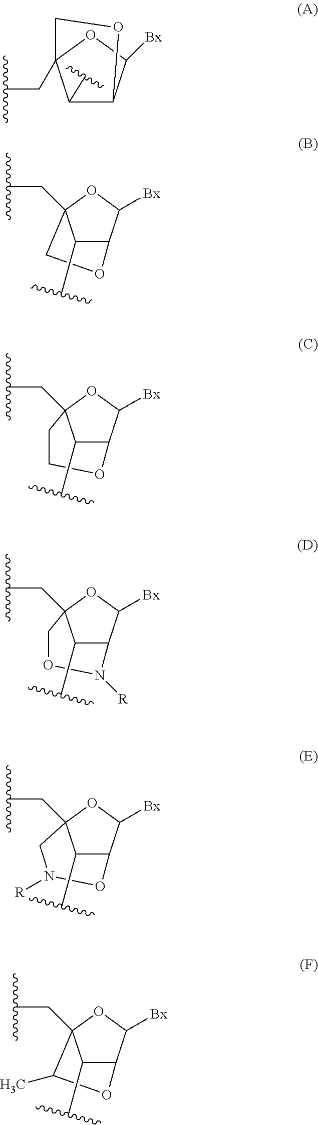

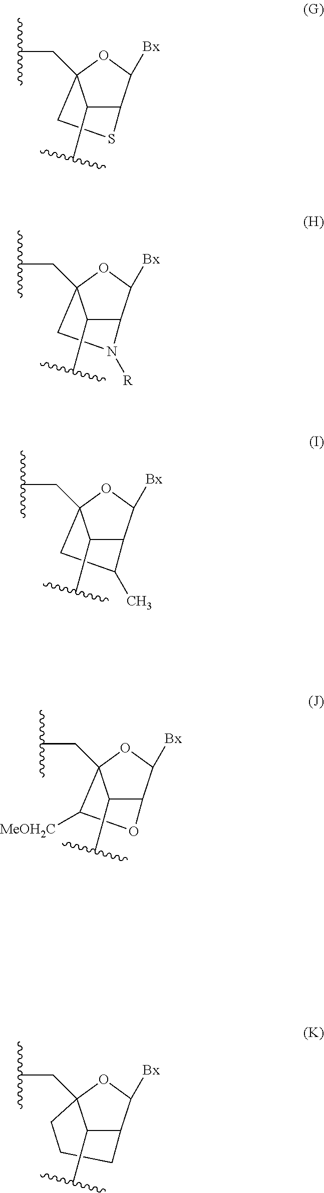

wherein Bx is a nucleobase moiety and R is, independently, H, a protecting group, or C₁-C₁₂ alkyl.

In certain embodiments, a 2′-modified nucleoside comprises a 2′-substituent group selected from F, OCF₃, O—CH₃ (also referred to as “2′-OMe”), OCH₂CH₂OCH₃ (also referred to as “2′-O-methoxyethyl” or “2′-MOE”), 2′-O(CH₂)₂SCH₃, O—(CH₂)₂—O—N(CH₃)₂, —O(CH₂)₂O(CH₂)₂N—(CH₃)₂, and O—CH₂—C(═O)—N(H)CH₃.

In certain embodiments, a 2′-modified nucleoside comprises a 2′-substituent group selected from F, O—CH₃, and OCH₂CH₂OCH₃.

In certain embodiments, a sugar-modified nucleoside is a 4′-thio modified nucleoside. In certain embodiments, a sugar-modified nucleoside is a 4′-thio-2′-modified nucleoside. A 4′-thio modified nucleoside has a β-D-ribonucleoside where the 4′-O replaced with 4′-S. A 4′-thio-2′-modified nucleoside is a 4′-thio modified nucleoside having the 2′-OH replaced with a 2′-substituent group. Suitable 2′-substituent groups include 2′-OCH₃, 2′-OCH₂CH₂OCH₃, and 2′-F.

In certain embodiments, a modified oligonucleotide comprises one or more internucleoside modifications. In certain such embodiments, each internucleoside linkage of a modified oligonucleotide is a modified internucleoside linkage. In certain embodiments, a modified internucleoside linkage comprises a phosphorus atom.

In certain embodiments, a modified oligonucleotide comprises at least one phosphorothioate internucleoside linkage. In certain embodiments, each internucleoside linkage of a modified oligonucleotide is a phosphorothioate internucleoside linkage.

In certain embodiments, a modified oligonucleotide comprises one or more modified nucleobases. In certain embodiments, a modified nucleobase is selected from 5-hydroxymethyl cytosine, 7-deazaguanine and 7-deazaadenine. In certain embodiments, a modified nucleobase is selected from 7-deaza-adenine, 7-deazaguanosine, 2-aminopyridine and 2-pyridone. In certain embodiments, a modified nucleobase is selected from 5-substituted pyrimidines, 6-azapyrimidines and N-2, N-6 and O-6 substituted purines, including 2 aminopropyladenine, 5-propynyluracil and 5-propynylcytosine.

In certain embodiments, a modified nucleobase comprises a polycyclic heterocycle. In certain embodiments, a modified nucleobase comprises a tricyclic heterocycle. In certain embodiments, a modified nucleobase comprises a phenoxazine derivative. In certain embodiments, the phenoxazine can be further modified to form a nucleobase known in the art as a G-clamp.

In certain embodiments, a modified oligonucleotide is conjugated to one or more moieties which enhance the activity, cellular distribution or cellular uptake of the resulting antisense oligonucleotides. In certain such embodiments, the moiety is a cholesterol moiety. In certain embodiments, the moiety is a lipid moiety. Additional moieties for conjugation include carbohydrates, peptides, antibodies or antibody fragments, phospholipids, biotin, phenazine, folate, phenanthridine, anthraquinone, acridine, fluoresceins, rhodamines, coumarins, and dyes. In certain embodiments, the carbohydrate moiety is N-acetyl-D-galactosamine (GalNac). In certain embodiments, a conjugate group is attached directly to an oligonucleotide. In certain embodiments, a conjugate group is attached to a modified oligonucleotide by a linking moiety selected from amino, azido, hydroxyl, carboxylic acid, thiol, unsaturations (e.g., double or triple bonds), 8-amino-3,6-dioxaoctanoic acid (ADO), succinimidyl 4-(N-maleimidomethyl) cyclohexane-1-carboxylate (SMCC), 6-aminohexanoic acid (AHEX or AHA), substituted C1-C10 alkyl, substituted or unsubstituted C2-C10 alkenyl, and substituted or unsubstituted C2-C10 alkynyl. In certain such embodiments, a substituent group is selected from hydroxyl, amino, alkoxy, azido, carboxy, benzyl, phenyl, nitro, thiol, thioalkoxy, halogen, alkyl, aryl, alkenyl and alkynyl.

In certain such embodiments, the compound comprises a modified oligonucleotide having one or more stabilizing groups that are attached to one or both termini of a modified oligonucleotide to enhance properties such as, for example, nuclease stability. Included in stabilizing groups are cap structures. These terminal modifications protect a modified oligonucleotide from exonuclease degradation, and can help in delivery and/or localization within a cell. The cap can be present at the 5′-terminus (5′-cap), or at the 3′-terminus (3′-cap), or can be present on both termini. Cap structures include, for example, inverted deoxy abasic caps.

### Certain Pharmaceutical Compositions

Provided herein are pharmaceutical compositions comprising a compound or modified oligonucleotide provided herein, and a pharmaceutically acceptable diluent. In certain embodiments, the pharmaceutically acceptable diluent is an aqueous solution. In certain embodiments, the aqueous solution is a saline solution. As used herein, pharmaceutically acceptable diluents are understood to be sterile diluents. Suitable administration routes include, without limitation, intravenous and subcutaneous administration. In certain embodiments, administration is intravenous administration. In certain embodiments, administration is subcutaneous administration. In certain embodiments, administration is oral administration.

In certain embodiments, a pharmaceutical composition is administered in the form of a dosage unit. For example, in certain embodiments, a dosage unit is in the form of a tablet, capsule, or a bolus injection.

In certain embodiments, a pharmaceutical agent is a modified oligonucleotide which has been prepared in a suitable diluent, adjusted to pH 7.0-9.0 with acid or base during preparation, and then lyophilized under sterile conditions. The lyophilized modified oligonucleotide is subsequently reconstituted with a suitable diluent, e.g., aqueous solution, such as water or physiologically compatible buffers such as saline solution, Hanks's solution, or Ringer's solution. The reconstituted product is administered as a subcutaneous injection or as an intravenous infusion. The lyophilized drug product may be packaged in a 2 mL Type I, clear glass vial (ammonium sulfate-treated), stoppered with a bromobutyl rubber closure and sealed with an aluminum overseal.

In certain embodiments, the pharmaceutical compositions provided herein may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents.

In some embodiments, the pharmaceutical compositions provided herein may contain additional materials useful in physically formulating various dosage forms of the compositions of the present invention, such as dyes, flavoring agents, preservatives, antioxidants, opacifiers, thickening agents and stabilizers; such additional materials also include, but are not limited to, excipients such as alcohol, polyethylene glycols, gelatin, lactose, amylase, magnesium stearate, talc, silicic acid, viscous paraffin, hydroxymethylcellulose and polyvinylpyrrolidone. In various embodiments, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the oligonucleotide(s) of the formulation. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes. Aqueous injection suspensions may contain substances that increase the viscosity of the suspension, such as sodium carboxymethyl cellulose, sorbitol, or dextran. Optionally, such suspensions may also contain suitable stabilizers or agents that increase the solubility of the pharmaceutical agents to allow for the preparation of highly concentrated solutions.

Lipid moieties have been used in nucleic acid therapies in a variety of methods. In one method, the nucleic acid is introduced into preformed liposomes or lipoplexes made of mixtures of cationic lipids and neutral lipids. In another method, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, a pharmaceutical composition provided herein comprise a polyamine compound or a lipid moiety complexed with a nucleic acid. In certain embodiments, such preparations comprise one or more compounds each individually having a structure defined by formula (Z) or a pharmaceutically acceptable salt thereof,

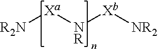

wherein each X^{a} and X^{b}, for each occurrence, is independently C₁₋₆ alkylene; n is 0, 1, 2, 3, 4, or 5; each R is independently H, wherein at least n+2 of the R moieties in at least about 80% of the molecules of the compound of formula (Z) in the preparation are not H; m is 1, 2, 3 or 4; Y is O, NR², or S; R¹ is alkyl, alkenyl, or alkynyl; each of which is optionally substituted with one or more substituents; and R² is H, alkyl, alkenyl, or alkynyl; each of which is optionally substituted each of which is optionally substituted with one or more substituents; provided that, if n=0, then at least n+3 of the R moieties are not H. Such preparations are described in PCT publication WO/2008/042973, which is herein incorporated by reference in its entirety for the disclosure of lipid preparations. Certain additional preparations are described in Akinc et al., Nature Biotechnology 26, 561-569 (1 May 2008), which is herein incorporated by reference in its entirety for the disclosure of lipid preparations.

In certain embodiments, a pharmaceutical composition provided herein is prepared using known techniques, including, but not limited to mixing, dissolving, granulating, dragee-making, levigating, emulsifying, encapsulating, entrapping or tableting processes.

In certain embodiments, a pharmaceutical composition provided herein is a solid (e.g., a powder, tablet, and/or capsule). In certain of such embodiments, a solid pharmaceutical composition comprising one or more oligonucleotides is prepared using ingredients known in the art, including, but not limited to, starches, sugars, diluents, granulating agents, lubricants, binders, and disintegrating agents.

In certain embodiments, a pharmaceutical composition provided herein is formulated as a depot preparation. Certain such depot preparations are typically longer acting than non-depot preparations. In certain embodiments, such preparations are administered by implantation (for example subcutaneously or intramuscularly) or by intramuscular injection. In certain embodiments, depot preparations are prepared using suitable polymeric or hydrophobic materials (for example an emulsion in an acceptable oil) or ion exchange resins, or as sparingly soluble derivatives, for example, as a sparingly soluble salt.

In certain embodiments, a pharmaceutical composition provided herein comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, a pharmaceutical composition provided herein comprises one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, a pharmaceutical composition provided herein comprises a sustained-release system. A non-limiting example of such a sustained-release system is a semi-permeable matrix of solid hydrophobic polymers. In certain embodiments, sustained-release systems may, depending on their chemical nature, release pharmaceutical agents over a period of hours, days, weeks or months.

Certain pharmaceutical compositions for injection are presented in unit dosage form, e.g., in ampoules or in multi-dose containers.

In certain embodiments, a pharmaceutical composition provided herein comprises a modified oligonucleotide in a therapeutically effective amount. In certain embodiments, the therapeutically effective amount is sufficient to prevent, alleviate or ameliorate symptoms of a disease or to prolong the survival of the subject being treated.

In certain embodiments, one or more modified oligonucleotides provided herein is formulated as a prodrug. In certain embodiments, upon in vivo administration, a prodrug is chemically converted to the biologically, pharmaceutically or therapeutically more active form of an oligonucleotide. In certain embodiments, prodrugs are useful because they are easier to administer than the corresponding active form. For example, in certain instances, a prodrug may be more bioavailable (e.g., through oral administration) than is the corresponding active form. In certain instances, a prodrug may have improved solubility compared to the corresponding active form. In certain embodiments, prodrugs are less water soluble than the corresponding active form. In certain instances, such prodrugs possess superior transmittal across cell membranes, where water solubility is detrimental to mobility. In certain embodiments, a prodrug is an ester. In certain such embodiments, the ester is metabolically hydrolyzed to carboxylic acid upon administration. In certain instances the carboxylic acid containing compound is the corresponding active form. In certain embodiments, a prodrug comprises a short peptide (polyaminoacid) bound to an acid group. In certain of such embodiments, the peptide is cleaved upon administration to form the corresponding active form.

In certain embodiments, a prodrug is produced by modifying a pharmaceutically active compound such that the active compound will be regenerated upon in vivo administration. The prodrug can be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

Additional administration routes include, but are not limited to, oral, rectal, transmucosal, intestinal, enteral, topical, suppository, through inhalation, intrathecal, intracardiac, intraventricular, intraperitoneal, intranasal, intraocular, intratumoral, intramuscular, and intramedullary administration. In certain embodiments, pharmaceutical intrathecals are administered to achieve local rather than systemic exposures. For example, pharmaceutical compositions may be injected directly in the area of desired effect (e.g., into the kidney).

### Certain Kits

The present invention also provides kits. In some embodiments, the kits comprise one or more compounds comprising a modified oligonucleotide disclosed herein. In some embodiments, the kits may be used for administration of the compound to a subject.

In certain embodiments, the kit comprises a pharmaceutical composition ready for administration. In certain embodiments, the pharmaceutical composition is present within a vial. A plurality of vials, such as 10, can be present in, for example, dispensing packs. In some embodiments, the vial is manufactured so as to be accessible with a syringe. The kit can also contain instructions for using the compounds.

In some embodiments, the kit comprises a pharmaceutical composition present in a pre-filled syringe (such as a single-dose syringes with, for example, a 27 gauge, ½ inch needle with a needle guard), rather than in a vial. A plurality of pre-filled syringes, such as 10, can be present in, for example, dispensing packs. The kit can also contain instructions for administering the compounds comprising a modified oligonucleotide disclosed herein.

In some embodiments, the kit comprised a modified oligonucleotide provided herein as a lyophilized drug product, and a pharmaceutically acceptable diluent. In preparation for administration to a subject, the lyophilized drug product is reconstituted in the pharmaceutically acceptable diluent.

In some embodiments, in addition to compounds comprising a modified oligonucleotide disclosed herein, the kit can further comprise one or more of the following: syringe, alcohol swab, cotton ball, and/or gauze pad.

### Certain Experimental Models

In certain embodiments, the present invention provides methods of using and/or testing modified oligonucleotides of the present invention in an experimental model. Those having skill in the art are able to select and modify the protocols for such experimental models to evaluate a pharmaceutical agent of the invention.

Generally, modified oligonucleotides are first tested in cultured cells. Suitable cell types include those that are related to the cell type to which delivery of a modified oligonucleotide is desired in vivo. For example, suitable cell types for the study of the methods described herein include primary or cultured cells.

In certain embodiments, the extent to which a modified oligonucleotide interferes with the activity of one or more microRNAs is assessed in cultured cells. In certain embodiments, inhibition of microRNA activity may be assessed by measuring the level of one or more of a predicted or validated microRNA-regulated transcript. An inhibition of microRNA activity may result in the increase in the microRNA-regulated transcript, and/or the protein encoded by the microRNA-regulated transcript (i.e., the microRNA-regulated transcript is de-repressed). Further, in certain embodiments, certain phenotypic outcomes may be measured.

Several animal models are available to the skilled artisan for the study of one or more microRNAs in models of human disease.

### Certain Quantitation Assays

In certain embodiments, microRNA levels are quantitated in cells or tissues in vitro or in vivo. In certain embodiments, changes in microRNA levels are measured by microarray analysis. In certain embodiments, changes in microRNA levels are measured by one of several commercially available PCR assays, such as the TaqMan® MicroRNA Assay (Applied Biosystems).

Modulation of microRNA activity with an anti-miR or microRNA mimic may be assessed by microarray profiling of mRNAs. The sequences of the mRNAs that are modulated (either increased or decreased) by the anti-miR or microRNA mimic are searched for microRNA seed sequences, to compare modulation of mRNAs that are targets of the microRNA to modulation of mRNAs that are not targets of the microRNA. In this manner, the interaction of the anti-miR with its target microRNA, or a microRNA mimic with its targets, can be evaluated. In the case of an anti-miR, mRNAs whose expression levels are increased are screened for the mRNA sequences that comprise a seed match to the microRNA to which the anti-miR is complementary.

Modulation of microRNA activity with an anti-miR compound may be assessed by measuring the level of a messenger RNA target of the microRNA, either by measuring the level of the messenger RNA itself, or the protein transcribed therefrom. Antisense inhibition of a microRNA generally results in the increase in the level of messenger RNA and/or protein of the messenger RNA target of the microRNA, i.e., anti-miR treatment results in de-repression of one or more target messenger RNAs.

### EXAMPLES

The following examples are presented in order to more fully illustrate some embodiments of the invention. They should in no way be construed, however, as limiting the broad scope of the invention.

Those of ordinary skill in the art will readily adopt the underlying principles of this discovery to design various compounds without departing from the spirit of the current invention.

### Example 1: The Role of miR-17 in PKD

miR-17 family members of the miR-17˜92 cluster of microRNAs are upregulated in mouse models of PKD. Genetic deletion of the miR-17˜92 cluster in a mouse model of PKD reduces kidney cyst growth, improves renal function, and prolongs survival (Patel et al., PNAS, 2013; 110(26): 10765-10770). The miR-17˜92 cluster contains 6 different microRNAs, each with a distinct sequence: miR-17, miR-18a, miR-19a, miR-19-b-1 and miR-92a-1.

The miR-17˜92 cluster includes two microRNAs, miR-17 and miR-20a, that are members of the miR-17 family of microRNAs. Each member of this family shares seed sequence identity, and varying degrees of sequence identity outside the seed region. The other members of the miR-17 family are miR-20b, miR-93, miR-106a, and miR-106b. miR-20b and miR-106a reside within the miR-106a˜363 cluster on the human X chromosome, and miR-93 and miR-106b reside within the miR-106b˜25 cluster on human chromosome 7. The sequences of the miR-17 family members are shown in Table 1.

TABLE 1
miR-17 family of microRNAs

| | SEQUENCE (5′ TO 3′) | SEQ ID |
|---|---|---|
| microRNA | seed region in bold | NO: |
| miR-17 | CAAAGUGCUUACAGUGCAGGUAG | 1 |
| miR-20a | UAAAGUGCUUAUAGUGCAGGUAG | 2 |
| miR-20b | CAAAGUGCUCAUAGUGCAGGUAG | 3 |
| miR-93 | CAAAGUGCUGUUCGUGCAGGUAG | 4 |
| miR-106a | AAAAGUGCUUACAGUGCAGGUAG | 5 |
| miR-106b | UAAAGUGCUGACAGUGCAGAU | 6 |

The anti-miR-17 compound RGLS4326 was discovered by screening a chemically diverse and rationally designed library of anti-miR-17 oligonucleotides for optimal pharmaceutical properties. RGLS4326 preferentially distributes to kidney and collecting duct-derived cysts, displaces miR-17 from translationally active polysomes, and de-represses multiple miR-17 mRNA targets including Pkd1 and Pkd2. Importantly, RGLS4326 attenuates cyst growth in human in vitro ADPKD models and multiple PKD mouse models after subcutaneous administration. A phase 1 single ascending dose (SAD) clinical trial of RGLS4326 in healthy volunteers was initiated in December 2017, followed by a phase 1 multiple ascending dose (MAD) clinical trial in healthy volunteers that was initiated in May 2018. A phase 1b clinical trial of RGLS4326 for the treatment of patients with autosomal dominant polycystic kidney disease (ADPKD) was initiated in October 2020.

Subsequent to the initiation of the phase 1 MAD clinical trial, nonclinical toxicology studies revealed central nervous system (CNS)-related findings, including abnormal gait, reduced motor activity, and/or prostration, at high doses of RGLS4326. To identify potential candidates for off-target pharmacology, a panel of 174 targets including G-protein coupled receptors, transporters, ion channels, nuclear receptors, and cytokine receptors was evaluated in vitro for possible interactions with RGLS4326. RGLS4326 was found to be an antagonist of the AMPA glutamate receptor, with a 50% inhibitory concentration (IC50) of 4.6 uM (14.2 ug/mL) based on ligand binding and a functional IC50 of 300-600 nM (0.9-1.8 ug/mL) based on patch clamp activity. AMPA receptors are ion channels on excitatory synapses in the CNS that mediate fast excitatory neurotransmission and, therefore, are key components of all neuronal networks. Such an interaction with the AMPA receptor could explain the CNS-mediated findings observed at high doses of RGLS4326 in nonclinical toxicology models.

### Example 2: Screen for Anti-miR-17 Compounds with Reduced AMPA Receptor Binding

RGLS4326 has the following sequence and chemical modification pattern: A_{S}G_{S}C_{M}A_{F}C_{F}U_{F}U_{M}U_{S}G_{S} where nucleosides followed by subscript “M” are 2′-O-methyl nucleosides, nucleosides followed by subscript “F” are 2′-fluoro nucleosides, nucleosides followed by subscript “S” are S-cEt nucleosides, each cytosine is a non-methylated cytosine, and all linkages are phosphorothioate linkages. Chemical modification and length variants of RGLS4326 were designed and screened to identify a compound that retains the potency and pharmacokinetic profile of RGLS4326 and exhibits reduced binding to the AMPA receptor (AMPA-R).

A library of compounds was designed with varying chemical modifications, nucleobase sequence, and length, relative to RGLS4326.

TABLE 2
anti-miR-17 Library

| | | | SEQ | |
|---|---|---|---|---|
| Compound | | Nucleobase | ID | |
| # | Chemical Notation | Sequence | NO | Length |
| RG-NG-1001 | A_{S}G_{S}C_{S}A_{S}C_{S}U_{S}U_{S}U_{S}G_{S} | AGCACUUUG | | 9 |
| RG-NG-1002 | A_{S}G_{S}C_{S}A_{S}C_{S}U_{S}U_{S}U_{S} | AGCACUUU- | | 8 |
| RG-NG-1003 | A_{S}G_{S}C_{M}A_{S}C_{M}U_{S}U_{M}U_{S}G_{S} | AGCACUUUG | | 9 |
| RG-NG-1004 | U_{S}A_{S}A_{S}G_{S}C_{S}A_{S}C_{S}U_{S}U_{S}U_{S}G_{S} | UAAGCACUUUG | 7 | 11 |
| RG-NG-1005 | U_{S}A_{S}A_{M}GC_{S}A_{S}C_{S}U_{M}U_{M}U_{S}G_{S} | UAAGCACUUUG | 8 | 11 |
| RG-NG-1006 | A_{L}G_{L}C_{L}A_{L}C_{L}T_{L}T_{L}T_{L}G | AGCACTTTG | | 9 |
| RG-NG-1007 | A_{S}G_{S}C_{F}A_{F}C_{F}U_{F}U_{F}U_{S}G_{S} | AGCACUUUG | | 9 |
| RG-NG-1008 | A_{S}G_{E}C_{M}A_{F}C_{F}U_{F}U_{M}T_{E}G_{S} | AGCACUUTG | | 9 |
| RG-NG-1009 | A_{E}G_{E}C_{M}A_{F}C_{F}U_{F}U_{M}T_{E}G_{E} | AGCACUUTG | | 9 |
| RG-NG-1010 | A_{E}G_{E}C_{M}A_{F}C_{F}U_{F}U_{M}U_{S}G_{S} | AGCACUUUG | | 9 |
| RG-NG-1011 | A_{L}G_{L}C_{M}A_{F}C_{F}U_{F}U_{M}U_{S}G_{S} | AGCACUUUG | | 9 |
| RG-NG-1012 | A_{S}G_{L}C_{M}A_{F}C_{F}U_{F}U_{M}T_{L}G_{S} | AGCACUUTG | | 9 |
| RG-NG-1013 | A_{S}A_{S}G_{S}C_{M}A_{F}C_{F}U_{F}U_{M}U_{S} | AAGCACUUU- | | 9 |
| RG-NG-1014 | A_{S}G_{S}C_{M}A_{F}C_{F}U_{F}U_{M}U_{S} | AGCACUUU- | | 8 |
| RG-NG-1015 | A_{S}G_{S}C_{M}A_{F}C_{F}U_{F}U_{M}U_{S}A_{S} | AGCACUUUA | | 9 |
| RG-NG-1016 | A_{S}G_{S}C_{M}A_{F}C_{F}U_{F}U_{M}U_{S}C_{S} | AGCACUUUC | | 9 |
| RG-NG-1017 | A_{S}G_{S}C_{M}A_{F}C_{F}U_{F}U_{M}U_{S}U_{S} | AGCACUUUU | | 9 |
| RG-NG-1018 | A_{F}C_{E}T_{E}G_{S}T_{E}AA_{S}G_{S}C_{M}A_{F}C_{F}U_{F}U_{M}U_{S}C_{S} | ACTGTAAGCACUUUC | 9 | 15 |
| RG-NG-1019 | A_{S}C_{S}TG_{S}U_{S}AA_{S}G_{S}C_{M}A_{F}C_{F}U_{F}U_{M}U_{S}C_{S} | ACTGUAAGCACUUUC | 10 | 15 |
| RG-NG-1020 | T_{E}G_{S}T_{E}AA_{S}G_{S}C_{M}A_{F}C_{F}U_{F}U_{M}U_{S}C_{S} | TGTAAGCACUUUC | 11 | 13 |
| RG-NG-1021 | T_{E}AA_{S}G_{S}C_{M}A_{F}C_{F}U_{F}U_{M}U_{S}C_{S} | TAAGCACUUUC | 12 | 11 |
| RG-NG-1022 | T_{E}A_{E}A_{S}G_{S}C_{M}A_{F}C_{F}U_{F}U_{M}U_{S}C_{S} | TAAGCACUUUC | 13 | 11 |
| RG-NG-1023 | A_{E}G_{S}C_{M}A_{F}C_{F}U_{F}U_{M}U_{S}G_{E} | AGCACUUUG | | 9 |
| RG-NG-1024 | A_{S}G_{S}C_{M}A_{F}C_{F}U_{F}U_{M}T_{E}G_{E} | AGCACUUTG | | 9 |
| RG-NG-1025 | A_{S}G_{S}C_{M}A_{F}C_{F}U_{F}U_{M}U_{S}G_{E} | AGCACUUUG | | 9 |
| RG-NG-1026 | A_{S}G_{S}C_{M}A_{L}C_{F}U_{F}U_{M}U_{S}C_{S} | AGCACUUUC | | 9 |
| RG-NG-1027 | A_{S}G_{S}C_{S}A_{F}C_{F}U_{F}U_{M}U_{S}C_{S} | AGCACUUUC | | 9 |
| RG-NG-1028 | A_{S}G_{S}C_{S}A_{L}C_{F}U_{F}U_{M}U_{S}C_{S} | AGCACUUUC | | 9 |
| RG-NG-1029 | A_{M}G_{L}C_{L}A_{L}C_{L}U_{M}T_{L}UC_{M} | AGCACUTUC | | 9 |
| RG-NG-1030 | A_{M}A_{M}G_{L}C_{L}A_{L}C_{L}U_{M}T_{L}UC_{M} | AAGCACUTUC | 14 | 10 |
| RG-NG-1031 | A_{M}A_{M}G_{L}C_{L}A_{L}C_{L}U_{M}T_{L}U_{M} | AAGCACUTU- | | 9 |

Nucleosides followed by subscript “M” are 2′-O-methyl nucleosides;
nucleosides followed by subscript “F” are 2′-fluoro nucleosides;
nucleosides followed by subscript “S” are S-cEt nucleosides;
nucleosides followed by subscript “E” are 2′-O-methoxyethyl (2′-MOE) nucleosides;
and
nucleosides followed by subscript “L” are LNA nucleosides.

The activity of anti-miR-17 compounds was evaluated in a radioligand binding assay which measured the binding of the [³H] AMPA ligand to the AMPA-R present on rat brain synaptic membranes, in the presence of increasing concentrations of anti-miR-17 compound. anti-miR-17 compounds with affinity for the AMPAR will bind to and compete with the binding of the [³H] AMPA ligand.

The assay was performed according to previously published methods (Honore et al., J Neurochem., 1982, 38(1):173-178; Olsen et al., Brain Res., 1987, 402(2):243-254). 5.0 nM of the ligand [³H] AMPA, 1.0 mM of the non-specific ligand L-Glutamic acid, and anti-miR compound at uM concentrations were incubated with synaptic membranes prepared from Wistar rat cerebral cortex for 90 minutes. The compounds shown in Table 2 were tested in three experiments. Anti-miRs targeted to microRNAs other than miR-17 were used as control compounds (RG5124 targeted to miR-33a; RG5365 targeted to let-7a; RG8093 targeted to miR-214). RGLS4326 and RG-NG-1001 were also tested in each experiment, as it was demonstrated to bind to and inhibit the activity of the AMPA-R. The amount of the [³H] AMPA ligand was quantitated by radioligand binding, and is shown in Tables 3, 4, and 5. As illustrated by the data, the compounds vary in their ability to inhibit binding of the radiolabeled ligand the AMPA-R.

TABLE 3
Inhibition of Ligand Binding to AMPA-R Experiment #1

| | | Nucleobase | | | | | |
|---|---|---|---|---|---|---|---|
| | SEQ | Sequence & | % Inhibition | | | | IC₅₀ |
| Compound | ID | Chemistry | @ uM | | | | |
| # | NO | (5′ to 3′) | 100 | 10 | 1 | 0.1 | (uM) |
| RG-NG-1001 | | A_{S}G_{S}C_{S}A_{S}C_{S}U_{S}U_{S}U_{S}G_{S} | 91.3 | 89.1 | 76.2 | 41.1 | 0.17 |
| RGLS4326 | | A_{S}G_{S}C_{M}A_{F}C_{F}U_{F}U_{M}U_{S}G_{S} | 104.1 | 93 | 55.5 | 17.6 | 0.70 |
| RG-NG-1002 | | A_{S}G_{S}C_{S}A_{S}C_{S}U_{S}U_{S}U_{S} | 24.0 | -16.9 | -2.0 | 0 | >100 |
| RG-NG-1003 | | A_{S}G_{S}C_{M}A_{S}C_{M}U_{S}U_{M}U_{S}G_{S} | 77.8 | 56.9 | 13.9 | 5.7 | 9.26 |
| RG-NG-1004 | 15 | U_{S}A_{S}A_{S}G_{S}C_{S}A_{S}C_{S}U_{S}U_{S}U_{S}G_{S} | 76.1 | 44.2 | 4.7 | -4.7 | 17.28 |
| RG-NG-1005 | 16 | U_{S}A_{S}A_{M}GC_{S}A_{S}C_{S}U_{M}U_{M}U_{S}G_{S} | 28.3 | 0.8 | -1.3 | 10.8 | >100 |
| RG-NG-1006 | | A_{L}G_{L}C_{L}A_{L}C_{L}T_{L}T_{L}T_{L}G_{L} | 93.9 | 68.9 | 35.3 | 6.7 | 2.77 |
| RG5124 | | A_{S}C_{S}A_{M}A_{F}U_{F}G_{F}C_{M}A_{S}C_{S} | -5.1 | -3.7 | 12.9 | 0.4 | >100 |
| | | (anti-miR-33a) | | | | | |
| RG5365 | | anti-let7a | -4 | 6.5 | -6.3 | 2.4 | >100 |
| RG8093 | | anti-miR-214 | -9.8 | -2 | -11.5 | -6.9 | >100 |

TABLE 4
Inhibition of Ligand Binding to AMPAR Experiment #2

| | SEQ | | % Inhibition | | | | |
|---|---|---|---|---|---|---|---|
| Compound | ID | Chemical | @ uM | | | | IC₅₀ |
| # | NO | Notation | 100 | 10 | 1 | 0.1 | (uM) |
| RG-NG-1001 | | A_{S}G_{S}C_{S}A_{S}C_{S}U_{S}U_{S}U_{S}G_{S} | 93.5 | 90.1 | 68.3 | 42.2 | 0.19 |
| RGLS4326 | | A_{S}G_{S}C_{M}A_{F}C_{F}U_{F}U_{M}U_{S}G_{S} | 96.8 | 93.6 | 73.1 | 31.5 | 0.27 |
| RG-NG-1002 | | A_{S}G_{S}C_{S}A_{S}C_{S}U_{S}U_{S}U_{S} | 24.6 | -2.1 | 4.6 | -5.5 | >100 |
| RG-NG-1007 | | A_{S}G_{S}C_{F}A_{F}C_{F}U_{F}U_{F}U_{S}G_{S} | 94.3 | 83.2 | 39.7 | 3.1 | 1.69 |
| RG-NG-1008 | | A_{S}G_{E}C_{M}A_{F}C_{F}U_{F}U_{M}T_{E}G_{S} | 59.6 | 22.1 | 13.6 | -22.4 | 56.58 |
| RG-NG-1009 | | A_{E}G_{E}C_{M}A_{F}C_{F}U_{F}U_{M}T_{E}G_{E} | 32.6 | 13 | 2.7 | -11.8 | >100 |
| RG-NG-1010 | | A_{E}G_{E}C_{M}A_{F}C_{F}U_{F}U_{M}U_{S}G_{S} | 86.4 | 70.1 | 31.3 | 0.7 | 3.44 |
| RG-NG-1011 | | A_{L}G_{L}C_{M}A_{F}C_{F}U_{F}U_{M}U_{S}G_{S} | 94 | 78.5 | 30.6 | -2.2 | 2.56 |
| RG-NG-1012 | | A_{S}G_{L}C_{M}A_{F}C_{F}U_{F}U_{M}T_{L}G_{S} | 88.3 | 69 | 38.5 | 9.9 | 2.56 |
| RG-NG-1013 | | A_{S}A_{S}G_{S}C_{M}A_{F}C_{F}U_{F}U_{M}U_{S} | 37.2 | 3.7 | -5.6 | -3.8 | >100 |
| RG-NG-1014 | | A_{S}G_{S}C_{M}A_{F}C_{F}U_{F}U_{M}U_{S} | 20.4 | 8.6 | -5.5 | 14.9 | >100 |
| RG-NG-1015 | | A_{S}G_{S}C_{M}A_{F}C_{F}U_{F}U_{M}U_{S}A_{S} | 19.9 | 6.5 | 11 | 2.3 | >100 |
| RG-NG-1016 | | A_{S}G_{S}C_{M}A_{F}C_{F}U_{F}U_{M}U_{S}C_{S} | 5.9 | 12.3 | 10.4 | 4.7 | >100 |
| RG-NG-1017 | | A_{S}G_{S}C_{M}A_{F}C_{F}U_{F}U_{M}U_{S}U_{S} | 8.1 | 4.3 | -1.3 | 0.2 | >100 |
| RG-NG-1026 | | A_{S}G_{S}C_{M}A_{L}C_{F}U_{F}U_{M}U_{S}C_{S} | 2.3 | -3.5 | -1.1 | -3.3 | >100 |
| RG-NG-1027 | | A_{S}G_{S}C_{S}A_{F}C_{F}U_{F}U_{M}U_{S}C_{S} | 3 | -2.2 | 2.3 | -2.2 | >100 |
| RG-NG-1028 | | A_{S}G_{S}C_{S}A_{L}C_{F}U_{F}U_{M}U_{S}C_{S} | 2.7 | 2 | 2.1 | 15.7 | >100 |
| RG-NG-1029 | | A_{M}G_{L}C_{L}A_{L}C_{L}U_{M}T_{L}UC_{M} | 38.4 | 7 | 15.9 | 0.9 | >100 |
| RG-NG-1030 | 17 | A_{M}A_{M}G_{L}C_{L}A_{L}C_{L}U_{M}T_{L}UC_{M} | 1.9 | 15 | 6.5 | 2.9 | >100 |
| RG-NG-1031 | | A_{M}A_{M}G_{L}C_{L}A_{L}C_{L}U_{M}T_{L}U_{M} | 16.7 | 2.7 | 1 | -4.3 | >100 |
| RG5124 | | A_{S}C_{S}A_{M}A_{F}U_{F}G_{F}C_{M}A_{S}C_{S} | 7.2 | -4.2 | 1.6 | -1.2 | >100 |
| | | (anti-miR-33a) | | | | | |

TABLE 5
Inhibition of Ligand Binding to AMPAR Experiment #3

| | SEQ | | % Inhibition | | | | |
|---|---|---|---|---|---|---|---|
| Compound | ID | | @ uM | | | | IC₅₀ |
| # | NO | Chemical Notation | 100 | 10 | 1 | 0.1 | (uM) |
| RG4047 | | A_{S}G_{S}C_{S}A_{S}C_{S}U_{S}U_{S}U_{S}G_{S} | 96.6 | 94.6 | 84.8 | 59.4 | 0.10 |
| RGLS4326 | | A_{S}G_{S}C_{M}A_{F}C_{F}U_{F}U_{M}U_{S}G_{S} | 97.2 | 89 | 55.8 | 14.6 | 0.78 |
| RG-NG-1018 | 18 | A_{E}C_{E}T_{E}G_{S}T_{E}AA_{S}G_{S}C_{M}A_{F}C_{F}U_{F}U_{M}U_{S}C_{S} | 12.3 | 1 | 8.1 | 3 | >100 |
| RG-NG-1019 | 19 | A_{S}C_{S}TG_{S}U_{S}AA_{S}G_{S}C_{M}A_{F}C_{F}U_{F}U_{M}U_{S}C_{S} | 21 | 15.6 | 17.2 | 12.9 | >100 |
| RG-NG-1020 | 20 | T_{E}G_{S}T_{E}AA_{S}G_{S}C_{M}A_{F}C_{F}U_{F}U_{M}U_{S}C_{S} | -7.9 | -23.3 | 6.6 | 17 | >100 |
| RG-NG-1021 | 21 | T_{E}AA_{S}G_{S}C_{M}A_{F}C_{F}U_{F}U_{M}U_{S}C_{S} | -1.5 | -11.5 | -10.9 | -2 | >100 |
| RG-NG-1022 | 22 | T_{E}A_{E}A_{S}G_{S}C_{M}A_{F}C_{F}U_{F}U_{M}U_{S}C_{S} | -10.9 | -7 | -5.4 | -23.5 | >100 |
| RG-NG-1023 | | A_{E}G_{S}C_{M}A_{F}C_{F}U_{F}U_{M}U_{S}G_{E} | 95.3 | 75.2 | 25.6 | 3.1 | 3.12 |
| RG-NG-1024 | | A_{S}G_{S}C_{M}A_{F}C_{F}U_{F}U_{M}T_{E}G_{E} | 35.2 | 6.5 | -4.7 | -17 | >100 |
| RG-NG-1025 | | A_{S}G_{S}C_{M}A_{F}C_{F}U_{F}U_{M}U_{S}G_{E} | 96.4 | 84.1 | 36.5 | -3.8 | 1.85 |
| RG5124 | | A_{S}C_{S}A_{M}A_{F}U_{F}G_{F}C_{M}A_{S}C_{S} | 23 | 16.1 | 9.3 | 5.1 | >100 |
| | | (anti-miR-33a) | | | | | |

To evaluate functional antagonism of anti-miR-17 oligonucleotides towards the AMPA-R, certain oligonucleotides were tested using the manual whole-cell patch clamp technique, which records membrane currents as a measure of AMPA-R activity.

Manual whole-cell patch clamp studies were performed by Metrion Biosciences (Cambridge, UK). Whole-cell voltage clamp experiments were performed at room temperature (18-21° C.) using an EPC 10 patch clamp amplifier using Patchmaster software (HEKA Elektronik). Glass patch pipettes were fabricated from borosilicate glass capillaries (Harvard Apparatus) to resistances between 1.4 and 2.5 MΩ. Membrane currents were recorded using the whole-cell patch clamp technique. ChanTest® GluA1/GluA4 EZCells were clamped at a holding potential of −80 mV and membrane currents elicited by 10 μM (S)-AMPA delivered using a VC38 perfusion system (ALA Scientific Instruments). The minimal current amplitude values were measured with each application of 10 μM (S)-AMPA. The fractional change of current amplitude produced by each concentration of compound was calculated relative to the control current (pre-compound) and expressed as percentage change (% inhibition) for each cell. The compounds tested are shown in Table 6. RGLS4326 was tested in a separate study from all other compounds in Table 6.

As shown in Table 6, relative to RGLS4326, compounds RG-NG-1015, RG-NG-1016, and RG-NG-1017 exhibited reduced functional antagonism towards AMPA-R based on the manual whole-cell patch clamp studies in human ChanTest® GluA1/GluA4 EZ-Cells.

TABLE 6
Functional Antagonism of AMPA-R
in Whole-Cell Patch Clamp Studies

| | | | %Inhibition on | |
|---|---|---|---|---|
| | | | membrane currents | |
| | | | evoked with 10 uM | |
| | Sequence & | | (s)-AMPA | |
| Compound | Chemistry | | (Mean + SD) | |
| ID | (5′-3′) | Target | 0.3 uM | 3 uM |
| RG4047 | A_{S}G_{S}C_{S}A_{S}C_{S}U_{S} | miR-17 | 80.5 ± 6.3 | 88.8 ± 4.5 |
| | U_{S}U_{S}G_{S} | | | |
| RGLS4326 | A_{S}G_{S}C_{M}A_{F}C_{F}U_{F} | miR-17 | 46.9 ± 9.8 | 87.4 ± 7.9 |
| | U_{M}U_{S}G_{S} | | | |
| RG-NG- | A_{S}G_{S}C_{M}A_{F}C_{F}U_{F} | miR-17 | 16.0 ± 5.0 | 39.5 ± 5.4 |
| 1015 | U_{M}U_{S}A_{S} | | | |
| RG-NG- | A_{S}G_{S}C_{M}A_{F}C_{F}U_{F} | miR-17 | 16.0 ± 6.2 | 27.1 ± 9.8 |
| 1016 | U_{M}U_{S}C_{S} | | | |
| RG-NG- | A_{S}G_{S}C_{M}A_{F}C_{F}U_{F} | miR-17 | 20.0 ± 6.3 | 28.2 ± 5.5 |
| 1017 | U_{M}U_{S}U_{S} | | | |
| RG5124 | A_{S}C_{S}A_{M}A_{F}U_{F}G_{F} | miR-33a | 14.1 ± 7.2 | 42.1 ± 9.4 |
| | C_{M}A_{S}C_{S} | | | |

### Example 3: Relationship Between Nucleobase Properties and AMPA-R Binding

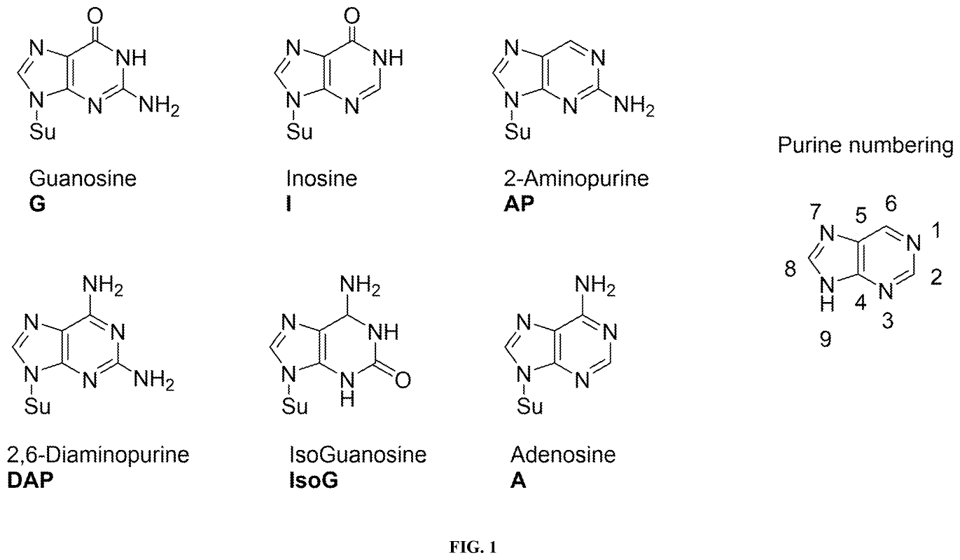

As illustrated by the AMPA-R binding and whole-cell patch clamp studies, the presence of guanosine at the 3′-terminus of an anti-miR-17 oligonucleotide, at the position complementary to the first nucleotide of miR-17, influences the functional antagonism of the AMPA-R. Like guanosine, adenosine is a purine, however adenosine did not inhibit the AMPA-R. Guanosine and adenosine are similar with regard to several properties except for hydrogen bonding, thus the differences in hydrogen bonding at positions 1, 2, and 6 of the purine base were evaluated. The purine nucleobases tested are shown in FIG. 1 and Table 7. In the “Purine Position” column of Table 7, “A” indicates a position of the purine that is hydrogen acceptor and “D” indicates a position of the purine that is a hydrogen donor. In the “Purine Position” column of Table 7, “N” indicates a neutral position that is neither a hydrogen acceptor or donor. Also tested were varying 2′-sugar moieties on the purine nucleobase, to evaluate the influence of 2′-sugar moiety chemistry on the ability of the purine nucleobase to inhibit the AMPA-R.

TABLE 7
Anti-miR-17 Compounds With Varying
Nucleobase and Sugar Moiety Chemistry

| | Nucleobase | 2′-Sugar | | | | | |
|---|---|---|---|---|---|---|---|
| | at | Moiety | | | | | |
| | 3-terminus | at | SEQ | Sequence & | Purine | | |
| Compound | (single letter | 3′- | ID | Chemistry | Position | | |
| ID | notation) | terminus | NO | (5′-3′) | # 6 | # 1 | # 2 |
| RG4047 | guanosine (G) | S-cEt | | A_{S}G_{S}C_{S}A_{S}C_{S}U_{S}U_{S}U_{S}G_{S} | A | D | D |
| RG-NG-1040 | guanosine (G) | S-cEt | 23 | C_{E}T_{E}G_{S}C_{E}A_{E}C_{E}T_{E}G_{S}T_{E}A_{D} | A | D | D |
| | | | | A_{S}G_{S}C_{M}A_{F}C_{F}U_{F}U_{M}U_{S}G_{S} | | | |
| RG-NG-1032 | guanosine (G) | S-cEt | 24 | G_{S}C_{E}A_{E}C_{E}T_{E}G_{S}T_{E}A_{D} | A | D | D |
| | | | | A_{S}G_{S}C_{M}A_{F}C_{F}U_{F}U_{M}U_{S}G_{S} | | | |
| RG4326 | guanosine (G) | S-cEt | | A_{S}G_{S}C_{M}A_{F}C_{F}U_{F}U_{M}U_{S}G_{S} | A | D | D |
| RG-NG-1033 | guanosine (G) | DNA | | A_{S}G_{S}C_{M}A_{F}C_{F}U_{F}U_{M}U_{S}G_{D} | A | D | D |
| RG-NG-1034 | guanosine (G) | 2′-O-methyl | | A_{S}G_{S}C_{M}A_{F}C_{F}U_{F}U_{M}U_{S}G_{M} | A | D | D |
| RG-NG-1035 | inosine (I) | S-cEt | | A_{S}G_{S}C_{M}A_{F}C_{F}U_{F}U_{M}U_{S}I_{M} | A | D | N |
| RG-NG-1036 | 2-aminopurine (N) | 2′-O-methyl | | A_{S}G_{S}C_{M}A_{F}C_{F}U_{F}U_{M}U_{S}N_{M} | N | A | D |
| RG-NG-1037 | 2,6-diaminopurine | 2′-O-methyl | | A_{S}G_{S}C_{M}A_{F}C_{F}U_{F}U_{M}U_{S}D_{M} | D | A | D |
| | (D) | | | | | | |
| RG-NG-1039 | isoguanine (F) | DNA | | A_{S}G_{S}C_{M}A_{F}C_{F}U_{F}U_{M}U_{SE}D | D | D | A |
| RG-NG-1038 | adenosine (A) | 2′-O-methyl | | A_{S}G_{S}C_{M}A_{F}C_{F}U_{F}U_{M}U_{S}A_{M} | D | A | N |
| RG-NG-1015 | adenosine (A) | S-cEt | | A_{S}G_{S}C_{M}A_{F}C_{F}U_{F}U_{M}U_{S}A_{S} | D | A | N |
| RG5124 | cytidine (C) | S-cEt | | A_{S}C_{S}A_{M}A_{F}U_{F}G_{F}C_{M}A_{S}C_{S} | | | |

The compounds were tested in the radioligand binding assay described herein, to determine the ability of the anti-miR-17 compounds to and compete with the binding of the [³H] AMPA ligand. As shown in Table 8, a correlation was observed between inhibition of ligand binding to the AMPA-R and the presence of a hydrogen bond acceptor at purine position #6 of the nucleobase at the 3′-terminus of the oligonucleotide. For example, compounds having guanosine or inosine at the 3′-terminus resulted in inhibition of ligand binding to the AMPA-R. Compounds with a 3′-terminal nucleobase having a hydrogen bond acceptor at purine position #6, for example RG-NG-1037 and RG-NG-1039, were less likely to inhibit ligand binding to the AMPA-K.

TABLE 8
Inhibition of Ligand Binding to AMPA-R

| | | 2′-Sugar | Purine | | | % Inhibition @ | | | | |
|---|---|---|---|---|---|---|---|---|---|---|
| | Nucleobase | Moiety at | Position | | | Concentration | | | | IC50 |
| ID | at 3-terminus | 3′-terminus | #6 | #1 | #2 | 100 uM | 10 uM | 1 uM | 0.1 uM | (uM) |
| RG4047 | guanosine | S-cEt | A | D | D | 102.9 | 99.3 | 81.3 | 41.9 | 0.15 |
| RG-NG-1040 | guanosine | S-cEt | A | D | D | 64.1 | 38.9 | 14.4 | 10.4 | 28.61 |
| RG-NG-1032 | guanosine | S-cEt | A | D | D | 92 | 78.3 | 43.8 | 24.3 | 1.17 |
| RGLS4326 | guanosine | S-cEt | A | D | D | 99.7 | 83.5 | 40.8 | −0.1 | 1.61 |
| RG-NG-1033 | guanosine | DNA | A | D | D | 97.3 | 96.5 | 80.9 | 44.9 | 0.13 |
| RG-NG-1034 | guanosine | 2′-O-methyl | A | D | D | 99.7 | 85.2 | 44.8 | 14.3 | 1.20 |
| RG-NG-1035 | inosine | S-cEt | A | D | N | 98.5 | 91.3 | 46.3 | 27.2 | 0.76 |
| RG-NG-1036 | 2-aminopurine | 2′-O-methyl | N | A | D | 78.1 | 36.2 | 11.3 | 19.2 | 18.61 |
| RG-NG-1037 | 2,6-diaminopurine | 2′-O-methyl | D | A | D | 10.7 | 2.4 | 9.2 | 11.1 | 100.00 |
| RG-NG-1039 | isoguanine | DNA | D | D | A | 30.2 | 35.8 | 27 | 27.3 | 100.00 |
| RG-NG-1038 | adenosine | 2′-O-methyl | D | A | N | 81.3 | 47.4 | 25.4 | 17.9 | 8.23 |
| RG-NG-1015 | adenosine | S-cEt | D | A | N | 5.6 | −7 | −10.3 | −0.8 | 100.00 |
| RG5124 | cytidine | S-cEt | | | | 10.1 | 4.4 | −0.6 | −8.6 | 100.00 |

### Example 4: In Vitro and In Vivo Potency of Anti-miR-17 Compounds

The in vitro potency of certain compounds was evaluated using a miR-17 luciferase sensor assay which uses a luciferase reporter vector for miR-17, with two fully complementary miR-17 binding sites in tandem in the 3′-UTR of the luciferase gene. HeLa cells were co-transfected with the luciferase reporter vector and an exogenous miR-17-expression vector that acted to repress the luciferase signal. HeLa cells were then individually treated with anti-miR-17 oligonucleotides at concentrations of 0.045, 0.137. 0.412, 1.23, 3.70, 11.1, 33.3, 100, and 300 nM. At the end of the 18- to 24-hour transfection period, luciferase activity was measured. RG5124 was included as a control compound. As shown in Table 9, these compounds inhibited miR-17 function and de-repressed miR-17 luciferases reporter activity with similar EC₅₀ values compared to RGLS4326 in vitro.

TABLE 9
Inhibition of miR-17 in Luciferase Assay

| | EC50 | Log2FC |
|---|---|---|
| Compound ID | (nM) | (37.5 nM) |
| RGLS4326 | 18.50 | 2.70 |
| RG-NG-1032 | <1 | 3.85 |
| RG-NG-1015 | 22.40 | 2.51 |
| RG-NG-1016 | 20.30 | 2.32 |
| RG-NG-1017 | 15.00 | 2.67 |
| RG5124 | n/a | 0.40 |

The activity of certain compounds was evaluated using a mouse miR-17 Pharmacodynamic-Signature (miR-17 PD-Sig), which consists of the expression of 18 unique miR-17 target genes normalized by six reference housekeeping genes, to provide an unbiased and comprehensive assessment of miR-17 activity. The mouse miR-17 PD-Sig score was the calculated average of the 18 genes' individual log 2 fold changes (normalized by six housekeeping genes) compared to mock transfection (Lee et al., Nat. Commun., 2019, 10, 4148).

As shown in Table 8, the tested oligonucleotides inhibited miR-17 function and de-repressed expression of multiple direct miR-17 target genes (as measured by miR-17 PD-signature) in normal and PKD kidney cell lines (both mouse and human) with similar EC₅₀ values compared to RGLS4326 in vitro. The PD-Sig for RGLS4326 in mIMCD3 cells (77.2, indicated by “*”) was not generated in this experiment; the value in Table 10 is that reported by Lee et al., Nat. Commun., 2019, 10, 4148. Blank cells in the table indicate that a compound was not tested in a particular cell line.

TABLE 10
miR-17 PD-Sig in Normal and PKD Cell Lines

| | | Derived | Anti-miR | | | | |
|---|---|---|---|---|---|---|---|
| Cell Line | Species | from | RGLS4326 | RG-NG-1015 | RG-NG-1016 | RG-NG-1017 | RG5124 |
| mIMCD3 | mouse | Normal | 77.2* | 168 | 146.3 | 134.5 | n/a |
| D52B5 | mouse | PKD | 92.8 | 82.4 | | 74.1 | 506.6 |
| HK-2 | human | Normal | 47.8 | 55.6 | | 49.2 | |
| WT9-7 | human | PKD | 17.3 | 21.2 | | 20.1 | |

In vivo potency was evaluated using the microRNA polysome shift assay (miPSA). This assay was used to determine the extent to which compounds directly engage the miR-17 target in the kidney in normal and PKD mice. The miPSA relies on the principle that active miRNAs bind to their mRNA targets in translationally active high molecular weight (HMW) polysomes, whereas the inhibited miRNAs reside in the low MW (LMW) polysomes. Treatment with anti-miR results in a shift of the microRNA from HMW polysomes to LMW polysomes. Thus, the miPSA provides a direct measurement of microRNA target engagement by a complementary anti-miR (Androsavich et al., Nucleic Acids Research, 2015, 44: e13).

Wildtype mice were administered a single dose of 0.3 mg/kg, 3 mg/kg, or 30 mg/kg. Kidney tissue was collected seven days later and subjected to the miPSA. The mean displacement score for each treatment is shown in Table 11 (PBS, n=17; RGLS326 30 mg/kg, n=10; all other treatments, n=4-5). The tested oligonucleotides displaced miR-17 from translationally active polysome (as measured by miPSA) in normal mouse kidneys.

TABLE 11
miPSA Displacement Scores

| | | | anti-miR Dose | | |
|---|---|---|---|---|---|
| | | | 0.3 m/kg | 3 mg/kg | 30 mg/kg |
| RG-NG-1015 | Mean | | 2.238 | 2.562 | 2.718 |
| | SEM | | 0.1242 | 0.1303 | 0.118 |
| RG-NG-1016 | Mean | | 1.892 | 2.263 | 1.134 |
| | SEM | | 0.1088 | 0.1716 | 0.5324 |
| RG-NG-1017 | Mean | | 2.077 | 2.292 | 2.793 |
| | SEM | | 0.1513 | 0.08974 | 0.09685 |
| RGLS4326 | Mean | | 1.846 | 2.247 | 2.362 |
| | SEM | | 0.1234 | 0.0478 | 0.1439 |
| Vehicle (PBS) | Mean | 0 | | | |
| | SEM | 0.06318 | | | |
| RG5124 | Mean | | | | 0.5935 |
| | SEM | | | | 0.1784 |

### Example 5: Efficacy of RG-NG-1015 in an Experimental Model of ADPKD

The efficacy of RG-NG-1015 was evaluated in the KspCre/Pkd1F/RC (Pkd1-KO) mouse model. Pkd1-KO is an orthologous ADPKD model that contains a germline hypomorphic Pkd1 mutation (the mouse equivalent of the human PKD1-R3277C (RC mutation) on one allele and loxP sites flanking Pkd1 exons 2 and 4 on the other allele. KspCre-mediated recombination was used to delete the floxed Pkd1 exons and produce a compound mutant mouse with a renal tubule-specific, somatic null mutation on one allele and a germline hypomorphic mutation on the other. This is an aggressive, but long-lived model of ADPKD (Hajarnis et al., Nat. Commun., 2017, 8, 14395).

On each of days 8, 10, 12, and 15 of age, sex-matched of Pkd1-KO mice were administered a subcutaneous injection of RGLS4326 at a dose of 20 mg/kg (n=8; 4 males and 4 females per treatment group), RG5124 at a dose of 20 mg/kg (n=8), or RG-NG-1015 at a dose of 20 mg/kg (n=8), or PBS (n=8). Mice were sacrificed at 18 days of age, and kidney weight, body weight, cyst index, serum creatinine level, and blood urea nitrogen (BUN) level were measured. BUN level is a marker of kidney function. A higher BUN level correlates with poorer kidney function, thus a reduction in BUN level is an indicator of reduced kidney injury and damage and improved function. Statistical significance was calculated by one-way ANOVA with Dunnett's multiple correction.

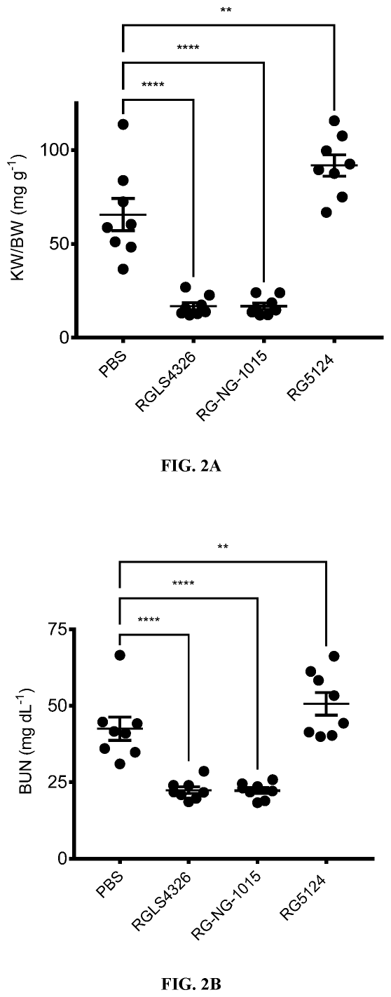

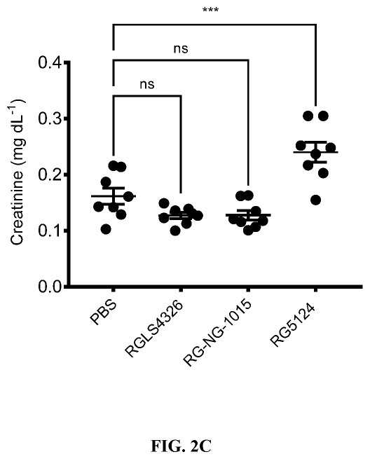

Results are shown in Table 12 and FIG. 2 (****=p<0.0001; ***=p<0.001; **=p<0.01; ns=not significant). The efficacy of RG-NG-1015 was similar to that of RGLS4326. The mean ratio of kidney weight to body weight (KW/BW ratio) was significantly lower in Pkd2-KO mice treated with RGLS4326 and RG1015, respectively, than the mean KW/BW ratio in Pkd2-KO mice administered PBS (FIG. 2A). Mean BUN levels were significantly reduced in Pkd2-KO mice treated with RGLS4326 and RG-NG-1015, respectively, compared to mice treated with PBS (FIG. 2B). Mean serum creatinine levels in Pkd2-KO mice were reduced in mice treated with RGLS4326 and RG-NG-1015, respectively, relative to mice treated with PBS, however the reduction was not statistically significant (FIG. 2C). Treatment with the control oligonucleotide, RG5124, did not reduce kidney weight to body weight ratio, serum creatinine, or serum BUN, demonstrating that the results observed with RGLS4329 and RG-NG-1015 were specific to the inhibition of miR-17.

TABLE 12
Efficacy of RG-NG-1015 in a Mouse Model of ADPKD

| | PBS | RGLS4326 | RG-NG-1015 | RG5124 |
|---|---|---|---|---|
| Mean | 65.61 | 16.81 | 16.83 | 91.75 |
| BW/KW Ratio | | | | |
| Mean Difference | | −48.8 | −48.79 | 26.14 |
| vs. PBS | | | | |
| Adjusted P- | | <0.0001 | <0.0001 | 0.0045 |
| Value | | | | |
| Mean | 42.48 | 22.41 | 22.26 | 50.61 |
| Serum BUN | | | | |
| Mean Difference | | −20.06 | −20.21 | +8.138 |
| vs. PBS | | | | |
| Adjusted P- | | <0.0001 | <0.0001 | 0.1142 |
| Value | | | | |
| Mean | 0.1619 | 0.1273 | 0.1279 | 0.2403 |
| Serum Creatine | | | | |
| Mean Difference | | −0.03463 | −0.03400 | +0.07838 |
| vs. PBS | | | | |
| Adjusted P- | | 0.1456 | 0.1557 | 0.0004 |
| Value | | | | |

### Example 6: Anti-miR-17 Compounds with Reduced Binding and Inhibition of AMPA-R Showed No CNS Toxicity in High Dose Studies

RG-NG-1015, RG-NG-1016, and RG-NG-1017 were tested in high-dose mouse toxicity studies. Each compound was tested in a single dose at 2000 mg/kg, and at escalating doses (100, 450, and 2000 mg/kg). As shown in Table 13, while escalating doses of RG-NG-1001 and RGLS4326 resulted in ataxia, lethargy, and in the case of RGLS4326, unconsciousness at the highest dose, no CNS-toxicity were observed for RG-NG-1015, RG-NG-1016, or RG-NG-1017.

TABLE 13
Anti-miR-17 Compounds and CNS-related findings

| | Mice/ | SC Dose | | |
|---|---|---|---|---|
| | group | (mg/kg/dose) | Schedule | CNS-related findings noted: |
| RG-NG-1001 | 8 | 100, 450, 2000 | QDx3 | Mild Body Scratching and Ataxia |
| | | | | observed at 100 and increase at 450 |
| | | | | mg/kg; Lethargy observed at 2000 mg/kg |
| RGLS4326 | 10 | 100, 450, 2000 | QDx4 | Ataxia and/or lethargy observed at 450 |
| | | | | mg/kg; Ataxia, lethargy and/or |
| | | | | unconsciousness observed at 2000 mg/kg |
| RG-NG-1015 | 7 | 2000 | Single | No |
| | 6 | 100, 450, 2000 | QDx4 | No |
| RG-NG-1016 | 10 | 2000 | Single | No |
| | 6 | 100, 450, 2000 | QDx4 | No |
| RG-NG-1017 | 10 | 2000 | Single | No |
| | 6 | 100, 450, 2000 | QDx4 | No |
| RG5124 | 7-10 | 100, 450, 2000 | QDx3 | Mild ataxia observed at 2000 mg/kg |

### Example 7: Maximum Tolerated Dose (MTD) Study and Comparative Dose Assessment of Different Compounds

Data from below studies further support that AMPA-R antagonism is responsible for CNS toxicity and mortality observed in previous toxicity studies of RGLS4326.

### Study 1: Maximum Tolerated Dose (MTD) Study and Comparative Dose Assessment of RG-NG-1017, RGLS4326 and RG-NG-1001

Compounds (RG-NG-1017, RGLS4326, RG-NG-1001) were evaluated in a pilot maximum tolerated dose (MTD) study (discussed below). RG-NG-1017, RGLS4326 and RG-NG-1001 were initially evaluated at 4 dose-levels each. RG-NG-1017 was included for evaluation as a non-AMPA-R binding compound, as compared to RGLS4326 and RG-NG-1001, which bind AMPA-R. C57Bl/6J male mice (Jackson Laboratories), age 6-7 weeks, were used in this study. Mice were assigned randomly to treatment groups, and the study was blinded. Animals were allowed to acclimate for no less than 5 days and housed on a 12 hr light/dark cycle (lights on 7:00 AM). No more than 4 mice were house in each cage in a ventilated cage rack system. The diet consisted of standard rodent chow and water ad libitum.

### MTD Pilot Study

The following parameters were used for this study:
1. Route(s) of administration: intracerebroventricular (ICV) dosing of RG-NG-1017, RG-NG-1001, RGLS4326
2. Dose Volume(s): 4 μL
3. Formulation(s): vehicle, Ca²⁺ and Mg²⁺ free dPBS
4. Dose Frequency: Once
5. Study duration: 8 Days
6. Number of Groups: 3
7. Number of animals per group: (2-4 each group)
8. Total number of animals: 54

For the ICV administration, mice were anesthetized and positioned for injections. The skin over the skull was incised, and a small hole was made in the skull above the target using a microdrill. The stereotactic coordinates were anteroposterior (AP), −0.4 mm; mediolateral (ML), +/−1.0-1.5 mm; dorsoventral (DV), −3.0 mm from the bregma for injection into both the right and left lateral cerebral ventricles (Hironaka et al, 2015). Animals were injected unilaterally with 4 μl into the right lateral cerebral ventricle. Compounds were injected over 1-2 min, and the needle was left in place for 0.5-1 min prior to withdrawal. The incision was closed with sutures, wound clips, or VetBond.

Following ICV treatment (Day 0), animals were monitored for 7 days in which daily health checks, body weight, and mortality was recorded. On Day 7, brain and kidney were collected and fixed (10% formalin) and stored pending histology.

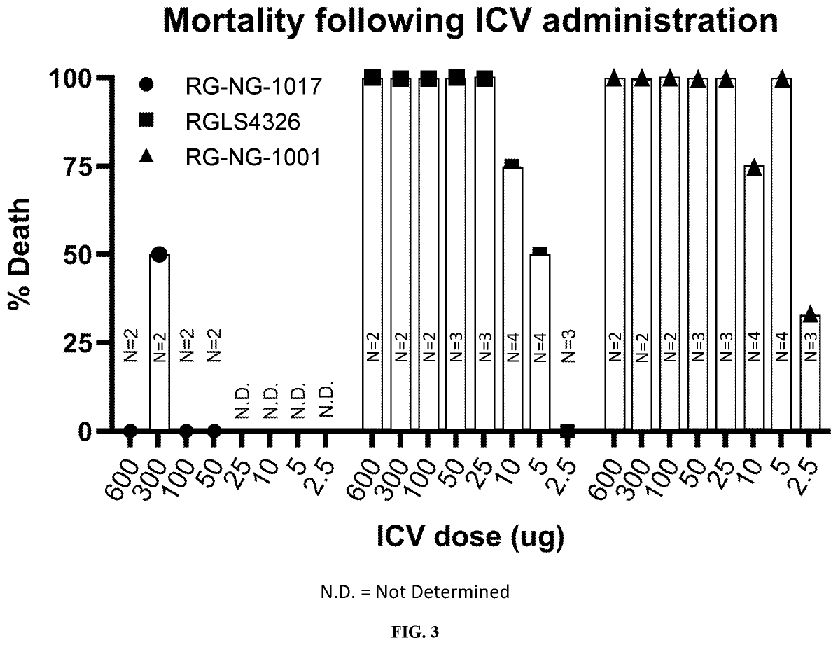

Results from the MTD study are shown in Table 14 and FIG. 3. All animal deaths were reported to occur within the first 5-8 hours post-ICV injection. Mice injected with 2.5 μg RG4326 were reported to display some immediate signs of respiratory distress and were provided heating pads. RG-NG-1017 (non-AMPA-R binding compound) was well-tolerated at high doses, with no established MTD for this compound (0 deaths at 600 μg, 100 μg, or 50 μg; 1 death at 300 μg). 100% mortality was observed at high doses for RG4326 and RG-NG-1001 (e.g., 600, 300, 100 μg), in addition to 100% mortality observed at 50 μg and 25 μg for both AMPA-R binding compounds. RG-NG-1001 MTD was not attained in this study, and was predicted to be under 2.5 μg. The MTD for RG4326 was predicted at ˜2.5<5.0 μg by ICV. All animals were reported to fully recover on Day 2 of observation.

TABLE 14
Summary Results from 7-day MTD Study

| | Mortality/total number of mice | | |
|---|---|---|---|
| Dose | | | RG-NG- |
| (μg) | RG-NG-1017 | RGLS4326 | 1001 |
| 600 | 0/2 | 2/2 | 2/2 |
| 300 | 1/2 | 2/2 | 2/2 |
| 100 | 0/2 | 2/2 | 2/2 |
| 50 | 0/2 | 3/3 | 3/3 |
| 25 | | 3/3 | 3/3 |
| 10 | | 3/4 | 3/4 |
| 5 | | 2/4 | 4/4 |
| 2.5 | | 0/3 | 1/3 |

### Maximum Tolerated Dose (MTD) Study for RGLS4326

A second MTD study for RGL4326 by ICV was conducted to assess dose selection for evaluating the compound in disease models (Table 15). In this study, a different mouse strain was evaluated (Swiss:Rjorl male mice, age 5 weeks, sourced from Janvier). Mice were placed under isoflurane anaesthesia (5% for induction and 2% for maintenance, under 100% O₂) and given 5 mg/kg s.c. carprofen (Rimadyl®). They were then placed in a stereotaxic frame. A midline sagittal incision was made in the scalp and a hole was drilled in the skull over the left lateral ventricle. A stainless-steel cannula (external diameter 0.51 mm) was placed stereotaxically into the left lateral ventricle at the following coordinates: +0.5 posterior to Bregma, L±0.7 mm, V=−2.7 mm. After a 2-minute delay to allow the brain tissue to slide over the cannula, 4 μL of a solution containing 0.625 mg/mL of RG4326 was slowly infused over 2 minutes. After infusion, the cannula was left in place for a further 5 minutes to prevent backflow of the solution along the cannula track. Mice were given 5 mg/kg s.c. carprofen (Rimadyl®) at 24 and 48 hours, after surgery. Mice were monitored during 3-7 days after surgery (starting 24 h after ICV administration) and their body weight was taken daily to check their health status. For mice monitored over 7 days, body weight was taken on Day 1 and on Day 7 after surgery to check their health status.

TABLE 15
Design of MTD Study for RGLS4326

| | Number | Treatment | | Concentration | Administration |
|---|---|---|---|---|---|
| Group | of animals | (RG4326) | Dose-level | (mg/mL) | Volume |
| 1 | 4 males | RG4326 (i.c.v.) | 3 mg/mouse | 0.75 mg/mL | 4 mL/mouse |
| 2 | 4 males | RG4326 (i.c.v.) | 4 mg/mouse | 1 mg/mL | 4 mL/mouse |
| 3 | 4 males | RG4326 (i.c.v.) | 5 mg/mouse | 1.25 mg/mL | 4 mL/mouse |
| 4 | 4 males | RG4326 (i.c.v.) | 7.5 mg/mouse | 1.875 mg/mL | 4 mL/mouse |

In Study 1, 6 mice were injected with 4 μL of a solution at 0.625 mg/mL (2.5 μg total per ICV). At the end of anesthesia, the mice remained lying on one side. They were quiet with some periods of scratching during the first hours after surgery. No toxic effects were observed at 24, 48 or 72 hours in the 6 mice administered. In Study 2, four mice were injected with 4 different doses of RGLS4326 (0.75, 1.0, 1.25 and 1.875 mg/mL, volume of 4 L). One mouse that received the highest dose (1.875 mg/mL, i.e., 7.5 μg/mouse) was found dead around 24 hours after ICV injection. All other mice were in good health, until the end of the pilot study (7 days after administration).

Combined results from Studies 1 and 2 demonstrated that RG4326 was generally well-tolerated in test subjects, but only at doses considerably lower than RG-NG-1017 (see FIG. 3).

Table 16 summarizes the MTD data for RGLS4326 for the Study 1 and 2 mouse models. Based on these results from Study 2, an MTD of ˜4 μg was predicted for RGLS4326 in the Swiss:Rjorl mouse strain.

TABLE 16
7-Day Survival Data for MTD Studies 1 and 2

| | |
|---|---|
| Dose | Dead/Total |
| (μg) | Mice |

| RGLS4326 | Study 1 | Study 2 |
|---|---|---|
| 600 | 2/2 | |
| 300 | 2/2 | |
| 100 | 2/2 | |
| 50 | 3/3 | |
| 25 | 3/3 | |
| 10 | 3/4 | |
| 7.5 | — | 1/4 |
| 5 | 2/4 | 0/4 |
| 4 | | 0/4 |
| 3 | | 0/4 |
| 2.5 | 0/3 | 0/6 |

In summary, compounds RG-NG-1017, RGLS4326 and RG-NG-1001 were evaluated across two MTD studies, demonstrating significant differences in tolerability between non-AMPA-R binding (RG-NG-1017) and AMPA-R binding compounds (RGLS4326, RG-NG-1001) (see FIG. 3). Despite 1 death at the ICV dose of 300 μg, an MTD was not established for RG-NG-1017, as no deaths occurred at the higher tested dose of 600 μg. Additionally, no impact on mortality was observed at doses of 100 and 50 μg for RG-NG-1017. By comparison, a clear impact on mortality was evident for the AMPA-R binding compounds RGLS4326 and RG-NG-1001, with no surviving animals across the tested dosing range of 25 μg to 600 μg. A trend for improved survival was seen at lower doses of RGLS4326 (10 μg), with 50% survival in RGLS4326 treated animals at 5 μg, and 100% survival at 2.5 μg. Similarly, in the case of RG-NG-1001 (which shows stronger AMPA-R binding compared to RGLS4326), 100% mortality was evident at the low dose of 5 μg, with a trend toward improved survival at 2.5 μg. The results for RGLS43426 from Study 1 were further confirmed in a second MTD study (Study 2), utilizing a different mouse strain. This study found that modest differences may exist for RGLS4326 tolerability between strains, with survival observed to only impact mice at the top dose of 7.5 μg versus at 5 μg in Study 1 using C57/Bl/6J. However, these results still support that MTD for the AMPA-R binding RGLS4326 occurs between ˜2.5 μg and 5-7.5 μg (depending on strain), as compared to a significantly higher MTD for non-AMPA-R binding RG-NG-1017 (at least >40-fold, or higher) (FIG. 3).

What is claimed is:

1. A compound comprising a modified oligonucleotide, wherein the modified oligonucleotide has the following structure in the 5′ to 3′ orientation:
(N)ₚ—(N″)—(N′)_{q}
p is from 7 to 24;
q is 0 or 1;
each N of (N)ₚ, independently, comprises a modified sugar moiety or an unmodified sugar moiety, and the nucleobase sequence of (N)ₚ is complementary to an equal-length portion of a microRNA, wherein (i) the nucleobase at position 1 of the microRNA is a uracil nucleobase and/or a cytosine nucleobase; (ii) the nucleobase at position 2 of the microRNA is a uracil nucleobase and/or a cytosine nucleobase; or (iii) the nucleobase at position 1 of the microRNA is a uracil and/or a cytosine nucleobase and the nucleobase at position 2 of the microRNA is a uracil nucleobase and/or a cytosine nucleobase;
N″ is a nucleoside comprising a modified sugar moiety or an unmodified sugar moiety, and the nucleobase of N″ is opposite position 2 of the microRNA;
wherein if q is 1, N′ is a nucleoside comprising a modified sugar moiety or an unmodified sugar moiety, and the nucleobase of N′ is opposite position 1 of the microRNA,
wherein if q is 0, the nucleobase of N″ is a uracil nucleobase, a cytosine nucleobase, or a purine nucleobase, provided that the purine nucleobase does not have a hydrogen bond acceptor at position 6,
wherein if q is 1, at least one nucleobase of N′ and N″ is a uracil nucleobase, a cytosine nucleobase, or a purine nucleobase, provided that the purine nucleobase does not have a hydrogen bond acceptor at position 6;
or a pharmaceutically acceptable salt thereof.

2. The compound of claim 1, wherein the nucleobase at position 1 of the microRNA is a uracil nucleobase, q is 1, and N′ is a uracil nucleobase, a cytosine nucleobase, or a purine nucleobase, provided that the purine nucleobase does not have a hydrogen bond acceptor at position 6.

3. The compound of claim 1 or 2, wherein the nucleobase at position 2 of the microRNA is a uracil nucleobase, and N″ is a uracil nucleobase, a cytosine nucleobase, or a purine nucleobase, provided that the purine nucleobase does not have a hydrogen bond acceptor at position 6.

4. The compound of claim 1 or 2, wherein the nucleobase at position 2 of the microRNA is a cytosine nucleobase, and N″ is a uracil nucleobase, a cytosine nucleobase, or a purine nucleobase, provided that the purine nucleobase does not have a hydrogen bond acceptor at position 6.

5. The compound of claim 3 or claim 4, wherein q is 0.

6. The compound of claim 1, wherein the nucleobase at position 1 of the microRNA is a cytosine nucleobase, q is 1, and N′ is a uracil nucleobase, a cytosine nucleobase, or a purine nucleobase, provided that the purine nucleobase does not have a hydrogen bond acceptor at position 6.

7. The compound of claim 1 or 6, wherein the nucleobase at position 2 of the microRNA is a cytosine nucleobase and N″ is a uracil nucleobase, a cytosine nucleobase, or a purine nucleobase, provided that the purine nucleobase does not have a hydrogen bond acceptor at position 6.

8. The compound of claim 1 or 6, wherein the nucleobase at position 2 of the microRNA is a uracil nucleobase and N″ is a uracil nucleobase, a cytosine nucleobase, or a purine nucleobase, provided that the purine nucleobase does not have a hydrogen bond acceptor at position 6.

9. The compound of claim 7 or claim 8, wherein q is 0.

10. The compound of any one of claims 1, 3-5, and 7-9, wherein the nucleobase of N″ is a purine nucleobase that does not have a hydrogen bond acceptor at position 6.

11. The compound of claim 10, wherein the nucleobase of N″ is selected from adenosine, 2-aminopurine, 2, 6-diaminopurine, and isoguanosine.

12. The compound of any one of claims 1 to 11, wherein the sugar moiety of N″ is not a 2′-O-methyl sugar.

13. The compound of any one of claims 1 to 11, wherein the sugar moiety of N″ is a 2′-O-methoxyethyl sugar or an S-cEt sugar.

14. The compound of any one of claims 1-4, 6-8, and 10-13, wherein the nucleobase of N′ is a purine nucleobase that does not have a hydrogen bond acceptor at position 6.

15. The compound of claim 14, wherein the nucleobase of N′ is selected from adenosine, 2-aminopurine, 2, 6-diaminopurine, and isoguanosine.

16. The compound of any one of claims 1-4, 6-8, and 10-15, wherein the sugar moiety of N′ is not a 2′-O-methyl sugar.

17. The compound of any one of claims 1-4, 6-8, and 10-15, wherein the sugar moiety of N′ is a 2′-O-methoxyethyl sugar or an S-cEt sugar.

18. The compound of any one of claims 1 to 17, wherein at least one internucleoside linkage is a phosphorothioate internucleoside linkage.

19. The compound of claim 18, wherein each internucleoside linkage is a phosphorothioate internucleoside linkage.

20. The compound of any one of claims 1 to 19, wherein p is 7, 8, 9, 10, 11, 12, 13, 14, 15, 16, 17, 18, 19, 20, 21, 22, 23, or 24.

21. The compound of any one of claims 1 to 20, wherein the nucleobase sequence of the modified oligonucleotide is at least 90%, is at least 95%, or is 100% complementary to the nucleobase sequence of the microRNA.

22. The compound of any one of claims 1 to 20, wherein the compound consists of the modified oligonucleotide.

23. The compound of any one of claims 1 to 22, wherein the pharmaceutically acceptable salt is a sodium salt.

24. A pharmaceutical composition comprising a compound of any one of claims 1 to 23 and a pharmaceutically acceptable diluent.

25. The pharmaceutical composition of claim 24, wherein the pharmaceutically acceptable diluent is an aqueous solution.

26. The pharmaceutical composition of claim 25, wherein the aqueous solution is a saline solution.

27. A pharmaceutical composition comprising a compound of any one of claims 1 to 23, which is a lyophilized composition.

28. A pharmaceutical composition consisting essentially of a compound of any one of claims 1 to 23 to in a saline solution.

29. A method for inhibiting the activity of a microRNA in a cell, comprising contacting the cell with a compound of any one of claims 1 to 23.

30. A method for inhibiting the activity of a microRNA in a subject, comprising administering to the subject a compound of any one of claims 1 to 23, or a pharmaceutical composition of any one of claims 24 to 28.

31. The method of claim 30, wherein the subject has a disease associated with the microRNA.

32. The method of claim 30 or 31, comprising administering a therapeutically effective amount of the compound.

33. The method of any one of claims 30 to 32, wherein the subject is a human subject.

34. A compound of any one of claims 1 to 23, or a pharmaceutical composition of any one of claims 27 to 28, for use in therapy.
